(19) United States
(12) Patent Application Publication
(10) Pub. No.: US 2025/0032815 A1
(43) Pub. Date: Jan. 30, 2025

(54) SYSTEMS AND METHODS FOR COOLING A PHOTOTHERAPY DEVICE

(71) Applicant: GlobaLaseReach, LLC, Manitowoc, WI (US)

(72) Inventors: Robert Nolan Hetz, Manitowoc, WI (US); Josh Zarecky, Fishers, IN (US); Jeff Stein, Minneapolis, MN (US); Scott Wichlacz, Manitowoc, WI (US); Joseph Pietrafitta, Manitowoc, WI (US)

(73) Assignee: GlobaLaseReach, LLC, Manitowoc, WI (US)

(21) Appl. No.: 18/704,584

(22) PCT Filed: Oct. 25, 2022
(86) PCT No.: PCT/US2022/047723

§ 371 Date: Apr. 25, 2024

Related U.S. Application Data
(60) Provisional application No. 63/271,774, filed on Oct. 26, 2021.

(51) Int. Cl. A61N 5/06; A61N 5/00

(52) U.S. Cl. CPC A61N 5/06; A61N 2005/005; A61N 2005/0626; A61N 2005/063; A61N 2005/0632

(57) ABSTRACT

A system for administering phototherapy comprising a cooling system, a coherent light generator, and a probe device. The cooling system configured to selectively circulate a coolant. The coherent light generator configured to produce a beam of coherent light. The probe device including an optical box at a distal end of a shaft, a fiber optic cable extending through the shaft to the optical box and configured to transmit the beam of coherent light from the coherent light generator through the optical box, and a coolant flow path extending through the shaft to the optical box and at least partially enveloping the fiber optic cable.

### CROSS-REFERENCE TO RELATED APPLICATIONS

This application claims the benefit of and priority to U.S. Provisional Patent Application No. 63/271,774, entitled “SYSTEMS AND METHODS FOR COOLING A PHOTOTHERAPY DEVICE,” filed Oct. 26, 2021, the entire disclosure of which is hereby incorporated by reference herein.

### BACKGROUND

The present disclosure relates to a device for delivering precision phototherapy, also known more specifically as photodynamic phototherapy or photobiomodulation therapy (“PBMT”). Light (photonic radiation) at certain wavelengths is more readily absorbed by molecules in certain tissues, identified as “chromophores,” which in turn can stimulate or retard certain metabolic processes. This can include stimulating, suppressing, or denaturing cellular tissues, interstitial tissues, and intracellular tissue components. The deliberate exposure of tissues to light for this purpose is known as “phototherapy,” “photobiomodulation therapy,” “low level light therapy,” “photodynamic therapy,” or “laser physiotherapy” in various applications.

### SUMMARY

One embodiment relates to a system for administering phototherapy comprising a cooling system, a coherent light generator, and a probe device. The cooling system is configured to selectively circulate a coolant. The coherent light generator is configured to produce a beam of coherent light. The probe device includes an optical box at a distal end of a shaft, a fiber optic cable extending through the shaft to the optical box and configured to transmit the beam of coherent light from the coherent light generator to the optical box, and a coolant flow path extending through the shaft to the optical box and at least partially enveloping the fiber optic cable.

In some embodiments, the coolant flow path includes a coolant inlet flow path configured to deposit the coolant into the optical box and a coolant outlet flow path extending through a proximal end of the optical box and at least partially enveloping the fiber optic cable. In some embodiments, the coolant outlet flow path comprises at least one vent port disposed adjacent to a diffusing lens arranged at or near the proximal end of the optical box, the coolant outlet flow path being configured to allow the coolant to directly cool the diffusing lens.

In some embodiments, the probe device further includes at least one of an internal temperature sensor or an external temperature sensor, and the cooling system is configured to selectively adjust at least one of a flow rate, a pressure, or a temperature of the coolant based on feedback from the at least one of the internal temperature sensor or the external temperature sensor. In some embodiments, the probe device includes the external temperature sensor and the external temperature sensor is arranged adjacent to an emission lens of the probe device.

In some embodiments, the coolant is a gas comprising one of CO2, nitrogen, or air.

In some embodiments, the coolant is a liquid and the coolant flow path extends through at least one of a sidewall of the optical box, a sidewall of a distal end of the probe device, or an emission lens of the probe device.

In some embodiments, the system further comprises an external chilling blanket configured to be slid over a distal end and the shaft of the probe device. In some embodiments, the external chilling blanket is cooled via one of a flowing coolant or a thermoelectric cooling element.

In some embodiments, the probe device further comprises one or more thermoelectric cooling elements embedded within or affixed to one or more of an emission lens, the optical box, or the distal end of the probe device.

Another embodiment relates to a system for administering phototherapy comprising a cooling system, a coherent light generator, and a probe device. The cooling system is configured to selectively circulate a coolant. The coherent light generator is configured to produce a beam of coherent light. The probe device includes an optical box at a distal end of a shaft and a coolant flow path extending through the shaft to the optical box. The optical box is configured to transmit the beam of coherent light from the coherent light generator. The coolant flow path includes a coolant inlet flow path configured to deposit the coolant into the optical box and a coolant outlet flow path configured to allow the coolant to exit the optical box.

In some embodiments, the probe device further includes at least one of an internal temperature sensor or an external temperature sensor, and the cooling system is configured to selectively adjust at least one of a flow rate, a pressure, or a temperature of the coolant based on feedback from the at least one of the internal temperature sensor or the external temperature sensor. In some embodiments, the probe device includes the external temperature sensor and the external temperature sensor is arranged adjacent to an emission lens of the probe device.

In some embodiments, the coolant flow path is an internal coolant flow path and the cooling system includes an external coolant flow path, and wherein at least a portion of at least one of the internal coolant flow path or the external coolant flow path includes an insulating sleeve.

In some embodiments, the probe device further comprises one or more thermoelectric cooling elements embedded within or affixed to one or more of an emission lens, the optical box, or the distal end of the probe device.

In some embodiments, the coolant outlet flow path comprises at least one vent port disposed adjacent to a diffusing lens arranged at or near a proximal end of the optical box, the coolant outlet flow path being configured to allow the coolant to directly cool the diffusing lens.

Another embodiment relates to a probe device for administering phototherapy. The probe device includes an optical box at a distal end of a shaft. The probe device further includes a fiber optic cable extending through the shaft to the optical box and configured to transmit a beam of coherent light from a coherent light generator through the optical box. The probe device further includes a coolant flow path extending through the shaft to the optical box and at least partially enveloping the fiber optic cable.

In some embodiments, the coolant flow path includes a coolant inlet flow path configured to deposit coolant into the optical box and a coolant outlet flow path extending through a proximal end of the optical box and at least partially enveloping the fiber optic cable. In some embodiments, the coolant outlet flow path comprises at least one vent port disposed adjacent to a diffusing lens arranged at or near the proximal end of the optical box, the coolant outlet flow path being configured to allow the coolant to directly cool the diffusing lens.

In some embodiments, the probe device further includes an external temperature sensor arranged adjacent to an emission lens of the probe device.

### BRIEF DESCRIPTION OF THE DRAWINGS

Further features, characteristics, and advantages of the present disclosure will become apparent to a person of ordinary skill in the art from the following detailed description of embodiments of the present disclosure, made with reference to the drawings annexed, in which like reference characters refer to like elements.

FIG. 1 is a schematic representation of a phototherapy system, according to an exemplary embodiment.

FIG. 2 is a partially exploded view of a probe device of the phototherapy system of FIG. 1, showing various internal and external components, according to an exemplary embodiment.

FIG. 3A is a top view of the probe device of FIG. 2, according to an exemplary embodiment.

FIG. 3B is a partial section view of the probe device of FIG. 3A, taken along line 3B-3B, according to an exemplary embodiment.

FIG. 3C is a detail section view of the distal end of the probe device of FIG. 3B, taken along detail line 3C-3C, according to an exemplary embodiment.

FIG. 4A is perspective view of various internal components of the probe device of FIG. 2, according to an exemplary embodiment.

FIG. 4B is partial detail view of a distal end of the probe device of FIG. 4A, taken along detail line 4B-4B, according to an exemplary embodiment.

FIG. 5A is partial view of a distal end of the probe device of FIG. 2, according to an exemplary embodiment.

FIG. 5B is a section view of the distal end of the probe device of FIG. 5A, taken along line 5B-5B, according to an exemplary embodiment.

FIG. 5C is a top view of an optical box of the probe device of FIG. 5A, shown without an emission lens, according to an exemplary embodiment.

FIG. 6 is a schematic representation of the phototherapy system of FIG. 1, showing various components of a cooling system, according to an exemplary embodiment.

FIG. 7 is a section view of a distal end of a probe device, according to an exemplary embodiment.

FIG. 8 is a section view of a distal end of a probe device, according to an exemplary embodiment.

FIG. 9A is an exploded view of a distal end of the probe device of FIG. 2, according to an exemplary embodiment.

FIG. 9B is a section view of the distal end of the probe device of FIG. 9A, taken along line 9B-9B, according to an exemplary embodiment.

FIG. 10 is a section view of a junction portion of the probe device of FIG. 2, according to an exemplary embodiment.

FIG. 11 is a schematic representation of another phototherapy system, showing various components of another cooling system, according to an exemplary embodiment.

FIG. 12A is a perspective view of various hardwiring components of a probe device of the phototherapy system of FIG. 11, according to an exemplary embodiment.

FIG. 12B is a detail view of the various hardwiring components of FIG. 12A, taken along detail line 12B-12B, according to an exemplary embodiment.

FIG. 12C is a detail view of the various hardwiring components of FIG. 12A, taken along detail line 12C-12C, according to an exemplary embodiment.

FIG. 13A is an axial view of the various hardwiring components of FIG. 12A, according to an exemplary embodiment.

FIG. 13B is a section view of the various hardwiring components of FIG. 13A, taken along section line 13B-13B, according to an exemplary embodiment.

FIG. 14 is a detailed section view of an optical box of the probe device of FIG. 13B, taken along detail line 14-14, according to an exemplary embodiment.

FIG. 15 is a detailed section view of an internal chamber of the probe device of FIG. 13B, taken along detail line 15-15, according to an exemplary embodiment.

FIG. 16A is a detailed section view of a proximal end section of an umbilical cable for connection to the probe device of FIG. 13B, taken along detail line 16A-16A, according to an exemplary embodiment.

FIG. 16B is a detailed section view of a support interface of the proximal end section of FIG. 16A, taken along detail line 16B-16B, according to an exemplary embodiment.

FIG. 16C is a detailed section view of a T-fitting of the proximal end section of FIG. 16A, taken along detail line 16C-16C, according to an exemplary embodiment.

FIGS. 17A and 17B depict a flowchart of a closed-loop cooling operating scheme for use with a phototherapy system, according to an exemplary embodiment.

FIG. 18 is a perspective view of a coolant flow control arrangement, according to an exemplary embodiment.

### DETAILED DESCRIPTION

The systems and methods described herein provide various phototherapy systems incorporating devices (e.g., probe devices) for the administration of photobiomodulation therapy (PBMT). In some instances, the probe devices may be used to provide transvaginal, transrectal, or any other type of PBMT to be applied topically or within an orifice of a patient. Further, the various systems and methods described herein may be incorporated into larger probe systems for the application of PBMT, as desired for a given application. Additionally, the various systems and methods described herein may be utilized in non-handheld devices, regardless of the size of such systems.

The phototherapy systems and methods descried herein include a unique coolant flow path that allows for an optical box and various optical components of the probe devices to be effectively cooled during operation. Specifically, the coolant flow path allows for a coolant (e.g., CO₂ gas) to be provided to and to cool various optical components of the optical box. The coolant then flows through various channels formed in the optical box and corresponding fiber connection components to effectively flow along and cool a diffusing lens (e.g., a ball lens), corresponding fiber connection components, and a fiber optic cable as the coolant is exhausted into a collection sleeve and ultimately out of the probe device.

Additionally, the systems and methods provide various cooling systems including a variety of control components configured to allow for the automatic adjustment of the cooling system flow rate, pressure, and/or temperature to provide adequate cooling to the probe devices during operation. This automatic adjustment of the cooling system flow rate, pressure, and/or temperature may be performed based on feedback from various internal and/or external temperature sensors and/or pressure sensors within the system. In some instances, the cooling system flow rate, pressure, and/or temperature may be controlled independently, collectively, or in any combination to provide cooling in response to various acquired temperature data.

It should be appreciated that the various cooling systems described herein may also be applied to a variety of other handheld devices, probe devices, and/or other therapy or monitoring systems or devices generally. In some instances, the cooling systems described herein may be utilized to cool a variety of handheld devices, probe devices, and/or other therapy or monitoring systems or devices configured to deliver various light-based therapies (e.g., PBMT, ablation, x-ray) to and/or allow for various patient monitoring (e.g., tomograms) of the esophagus, the rectum, the colon, bronchi, tumors (e.g., prostate tumors), and/or a variety of other anatomical features of the patient.

For example, in some instances, the cooling systems described herein may be utilized to circulate coolant within a transesophageal probe having one or more temperature sensors. In some instances, the cooling systems described herein may be utilized to circulate coolant within a PBMT treatment cylinder system having one or more temperature sensors. For example, in some instances, the handheld devices, probe devices, and/or other systems or devices cooled by the cooling system may be any of the systems and devices described in detail in U.S. Pat. No. 11,318,323, filed Aug. 21, 2020, the entirety of which is incorporated by reference herein.

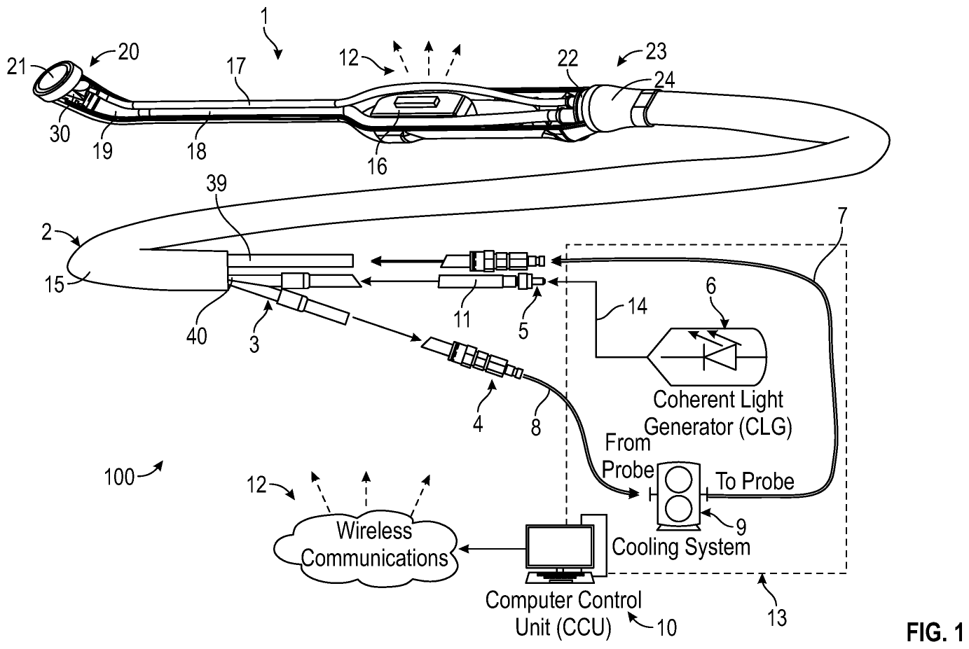

For example, shown in FIG. 1 is a phototherapy system 100, according to an exemplary embodiment. The phototherapy system 100 includes a probe device 1 configured to provide photobiomodulation therapy (PBMT) to a patient. In some instances, the probe device 1 is a handheld probe device. The probe device 1 is connected to various external components of the phototherapy system 100 via an umbilical cable 2.

The probe device 1 may be connected to a coherent light generator (CLG) 6 and a cooling system 9. For example, as illustrated in FIG. 1, the umbilical cable 2 may include a coolant inlet tube 39 and a coolant or return outlet tube 40 configured to collectively provide coolant to and receive coolant from the probe device 1. As illustrated, the coolant outlet tube 40 of the umbilical cable 2 may be split into a first channel and a second channel by a wye or “Y” junction 3 at a proximal end of the umbilical cable 2.

The first channel includes a coolant connection component 4 configured to fluidly couple the coolant outlet tube 40 with a cooling system inlet tube 8 to provide “hot” coolant (i.e., coolant that has been used to cool components of the probe device 1) from the probe device 1 to the cooling system 9. In some instances, the coolant connection component 4 is a quick-release type connector that automatically seals itself when not connected. For example, in some instances, the quick-release type connector is a dry-break quick disconnect pneumatic connector.

The second channel includes a fiber optic cable (FOC) connection component 5 configured to allow for a fiber optic cable (FOC) 14 to be fed from the CLG 6, into and through the coolant outlet tube 40, and into the probe device 1. In some instances, the FOC connection component 5 is a SubMinature version A (SMA) type connector configured for direct connection to an output of the CLG 6. Additionally, in some instances, the FOC 14 is configured to pass into a protective sheath 11 arranged between the FOC connection component 5 and the wye junction 3. The FOC 14 is configured to transmit therapeutic light energy from the CLG 6, through the FOC connection component 5, through the coolant outlet tube 40, and into the probe device 1, as will be further discussed below. Specifically, the FOC 14 is merged with the coolant outlet tube 40 via the wye junction 3 at the proximal end of the umbilical cable 2, such that the FOC 14 is coaxially arranged within the coolant outlet tube 40.

The cooling system 9 is configured to cool the “hot” coolant received from the probe device 1 via the cooling system inlet tube 8 and to provide the “cold” coolant (i.e., coolant that has been cooled via the cooling system 9) to a coolant inlet tube 39 of the probe device 1 within the umbilical cable 2 via a cooling system outlet tube 7. In some instances, the coolant utilized in the phototherapy system 100 may be a gas, such as CO₂ gas. In some other instances, various other coolants may be utilized within the phototherapy system 100. For example, in some instances, the coolant utilized in the phototherapy system 100 may be various other gases, such as chilled air or nitrogen. In some other instances, the coolant utilized in the phototherapy system 100 may be a liquid, such as water. In some instances, if liquid water is used as a coolant, the liquid water may be used by itself, distilled, or mixed with another substance (e.g., alcohol) to allow the coolant to flow at sub-zero (Celsius) temperatures without freezing. In some instances, the coolant (e.g., CO₂ gas) utilized in the phototherapy system 100 may be selected such that the coolant does not absorb any or nearly any of the light (e.g., infrared or near infrared light) delivered by the probe device 1, such that the coolant can be administered through the coolant flow path without the light being affected.

The coolant inlet tube 39 is configured to carry the “cold” coolant from the cooling system outlet tube 7 of the cooling system 9 received via a coolant connection component 4 (similar to the coolant connection component 4 discussed above) at the proximal end of the umbilical cable 2.

In some instances, the coolant connection components 4 connecting the coolant outlet tube 40 with the cooling system inlet tube 8 and the coolant inlet tube 39 with the cooling system outlet tube 7 are removably attached to bulkhead connections arranged on an external surface of an enclosure 13 (e.g., a cabinet, a cart, any other suitable enclosure) and then to the cooling system 9 via the cooling system outlet and inlet tubes 7, 8. In other instances, the coolant connection components 4 are connected directly to the cooling system 9.

The umbilical cable 2 comprises a protective sheath 15 enveloping both the coolant inlet tube 39 and the coolant outlet tube 40 having the FOC 14 disposed therein. The protective sheath 15 constitutes the external surface of the umbilical cable 2 and extends for the length of the umbilical cable 2 from a proximal end of the umbilical cable 2 to a proximal end 23 of the probe device 1. The umbilical cable 2 is further connected to the proximal end 23 of the probe device 1 and is supported and protected by a strain relief collar 24.

The probe device 1 includes a tubing junction 22 at the proximal end 23 of the probe device 1. The tubing junction 22 provides pneumatic tubing connections between the coolant inlet and outlet tubes 39, 40 from the umbilical cable 2 and internal coolant inlet and outlet tubes 17, 18. For example, the probe device 1 includes an internal coolant inlet tube 17 configured to carry coolant received from the cooling system 9 from the proximal end 23 of the probe device 1 to an optical box 30 at a distal end 20 of the probe device 1.

A collection sleeve 19 is configured to capture coolant exhausted from the optical box 30 at the distal end 20. From the collection sleeve 19, the coolant is then carried away from the distal end 20 of the probe device 1 via an internal coolant outlet tube 18 to the probe's proximal end 23 and then back to the cooling system 9 via the coolant outlet tube 40. Accordingly, heat pulled by the coolant from the optical box 30 and other components of the probe device 1 is effectively routed out of the probe device 1 and back to the cooling system 9 to be cooled and returned to the probe device 1 for continuous cooling of the various components of the probe device 1.

In some instances, various components of the phototherapy system 100 (e.g., the probe device 1, the CLG 6, the cooling system 9) are configured to be controlled via a computer control unit (CCU) 10. In some instances, the CCU may be located within and/or on the enclosure 13. The CCU 10 may be configured to communicate wirelessly with the various components of the phototherapy system 100 via a wireless network interface 12. For example, in some instances, the CCU 10 may be able to wirelessly communicate with integrated electronics 16 of the probe device 1 to control various functionality of the probe device 1.

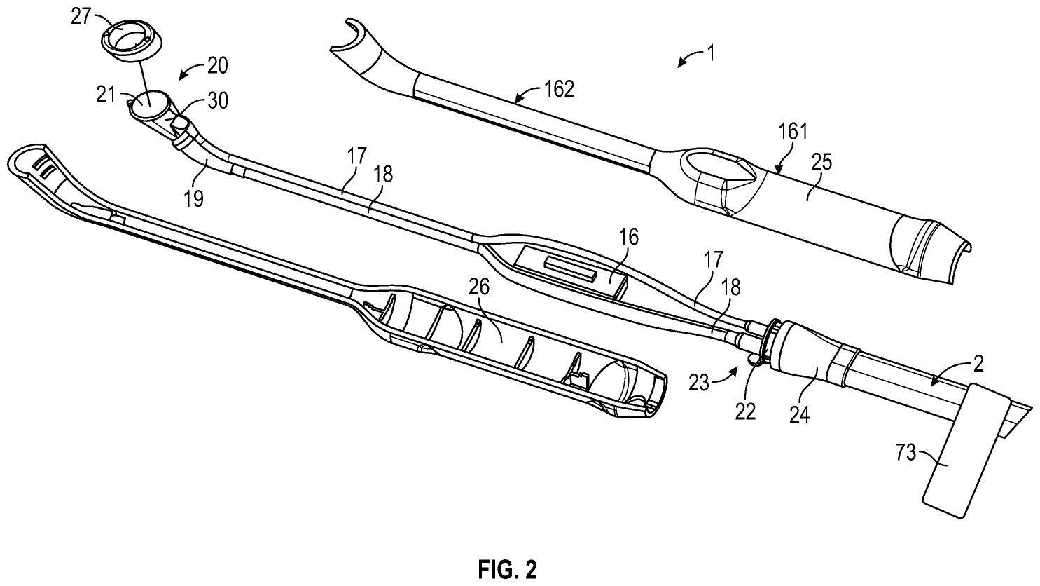

Referring now to FIG. 2, a partially exploded view of the probe device 1 is shown, illustrating various internal and external components and interfaces, according to an exemplary embodiment. For example, the probe device 1 includes a variety of internal elements, including the integrated electronics 16, the internal coolant inlet tube 17, the internal coolant outlet tube 18, the collection sleeve 19, and the optical box 30. The probe device 1 may further include a variety of additional internal elements, as will be discussed below.

In some instances, these internal elements of the probe device 1 may be encapsulated by one or more cover pieces collectively forming an enclosure. For example, in some instances, the probe device 1 may include a top cover 25, a bottom cover 26, and an end cap 27. However, it should be appreciated that, in some other instances, the enclosure may be formed differently. For example, in some embodiments, the enclosure may be formed by left and right halves. As shown in FIG. 2, in some embodiments, the end cap 27 may be on the distal end 20. In some other embodiments, the end cap 27 may alternatively be on the proximal end 23 of the probe device 1. In yet some other embodiments, the enclosure may be formed without utilizing an end cap 27.

In some instances, the enclosure may be sealed to prevent external fluids, gases, and/or other contaminants from entering the probe device 1. For example, the enclosure may be sealed using a variety of suitable methods including, but not limited to, epoxy or other adhesives, mechanical seals (e.g., an o-ring or other suitable gasket), interlocking features, or any other combination of geometries or added materials configured to create a seal between the various components of the enclosure. Additionally, in some instances, the tubing junction 22 at the proximal end 23 of the probe device 1 further facilitates sealing between the top cover 25, the bottom cover 26, and the umbilical cable 2 at the strain relief collar 24.

As shown in FIG. 2, in some instances, a larger-diameter section or grip portion 161 of the probe device 1, near the proximal end 23 of the probe device 1, is ergonomically contoured as a handle to be held by the user to manipulate the probe during therapeutic treatment. Adjacent the grip portion 161, a longer, smaller-diameter section or shaft 162 of the enclosure between the proximal end 23 and the distal end 20 allows for variable depth of insertion and easy rotation of the probe device 1 within the patient during treatment. At the distal end of the shaft 162, the distal end 20 of the probe device 1 is flared outward to accommodate the optical box 30 and an optical window lens 21.

The optical window lens 21 is an external interface from which collimated, diffuse, focused, and/or coherent therapeutic light is emitted. Accordingly, the optical window lens 21 serves as the patient tissue interface for the probe device 1. In some instances, the optical window lens 21 may be sized and integrated within the optical box 30 and enclosure end cap 27 to create an effective optical window (e.g., a phototherapy treatment window) of approximately 2.0 cm. In other instances, the optical window lens 21 may be sized and or integrated differently to create various other effective optical window sizes.

As shown in FIG. 2, in some instances, the umbilical cable 2 may include a handle 73 located on the exterior surface of the umbilical cable to facilitate easy manipulation of the umbilical cable 2 by the user with one hand while their other hand is holding and manipulating the probe device 1. In some instances, the handle 73 is configured to slide on the surface of the umbilical cable 2 along at least a portion of the length of the umbilical cable 2 to facilitate ergonomic positioning and use of the handle 73. Additionally, in some instances, the handle 73 is configured to rotate about the axis of the umbilical cable 2.

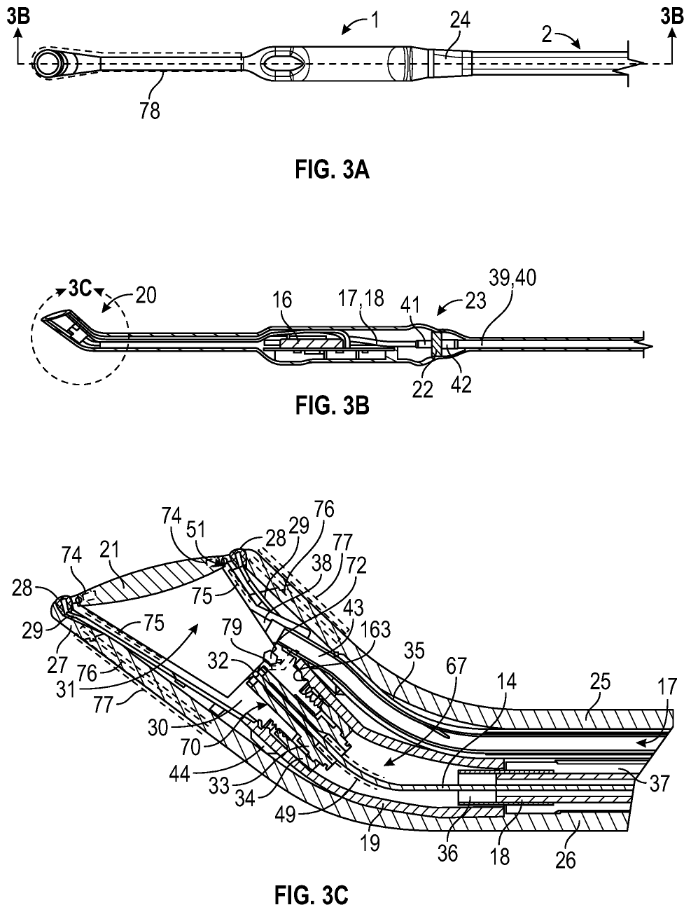

Referring now to FIGS. 3A-3C, a top view (FIG. 3A), a side section view (FIG. 3B) and a detail section view (FIG. 3C) of the probe device 1 are shown, according to various example embodiments. As best shown in FIG. 3B, the inlet and outlet tubes 39, 40 from the umbilical cable 2 are pneumatically connected to the internal coolant inlet and outlet tubes 17, 18 within the probe device 1 via the tubing junction 22. The internal coolant inlet and outlet tubes 17, 18 and the external inlet and outlet tubes 39, 40 are secured at the tubing junction 22 via internal tube end connections 41 and external tube end connections 42 to the enclosure (e.g., the top cover 25 and the bottom cover 26) of the probe device 1 at the proximal end 23 of the probe device 1. The tube end connections 41, 42 may comprise one or more joining configurations or methods including, but not limited to barbed and compression tube fittings, epoxy, glue or other adhesive bonding, and/or press fit or shrink fit interfaces. The strain relief collar 24 is configured to protect the external tube end connections 42 during use and manipulation of probe device 1 to prevent damage or failure of the proximal end 23 connection to the umbilical cable 2. In some embodiments, the external inlet and outlet tubes 39, 40 and the internal coolant inlet and outlet tubes 17, 18 can be continuous sections of tubing between the cooling system 9 and the probe device 1, such that the intermediate tubing connections 41, 42 may be omitted.

The distal end 20 of the probe device 1 is where therapeutic light energy is emitted (e.g., via the optical window lens 21) for the application of the photobiomodulation therapy. Specifically, the FOC 14 is carried within the coolant outlet tube 40, through the umbilical cable 2 and the internal coolant outlet tube 18, and terminates near the distal end 20 of the probe device 1 within the optical box 30 (as best shown in FIG. 3C). The distal end of the FOC 14 within the optical box 30 is retained within a fiber ferrule 33. For example, in some instances, the FOC 14 is adhered to or otherwise fixed within the fiber ferrule 33 using epoxy or other suitable methods.

In some instances, the probe device 1 further includes a stress sleeve 49 extending from and beyond a proximal end of the fiber ferrule 33 to support and protect the FOC 14 within the collection sleeve 19. In some instances, in lieu of directly securing the FOC 14 within the fiber ferrule 33, the stress sleeve 49 may be crimped onto the FOC 14 and is secured within the fiber ferrule 33.

The fiber ferrule 33 may be located in a controlled position and orientation by a fiber ferrule bore 70 formed within the optical box 30. For example, the fiber ferrule 33 may be coupled to and retained within the fiber ferrule bore 70 of the optical box 30 by a fiber retention nut 34. In some instances, the fiber ferrule 33 is arranged such that the distal end of the FOC 14 (e.g., where light is emitted from the FOC 14) is concentric and adjacent to a diffusing lens 32. As illustrated, the diffusing lens 32 may be a diffusing ball lens.

Accordingly, light emitted from the distal end of the FOC 14 is emitted incident upon the diffusing lens 32 (e.g., the ball lens), is diffracted through an optical box cavity 31 within the optical box 30, and is then collimated by the optical window lens 21 for delivery to patient tissue. In some instances, the surfaces of the optical box cavity 31 within the optical box 30 may be reflective to facilitate light reflection, thereby maximizing therapeutic light energy delivered to patient tissue.

The probe device 1 further includes external temperature sensors 28 located on the anterior and posterior sides of the distal end 20 of the probe device 1, adjacent to the optical window lens 21. In some instances, the external temperature sensors 28 may be arranged within the end cap 27. In some other instances, the external temperature sensors 28 may be arranged within other enclosure components depending on implementation of the embodiment. In any case, the external temperature sensors 28 are configured to be placed in contact with the patient tissue during treatment. Additionally, the probe device 1 includes one or more internal temperature sensors 72. As illustrated in FIG. 3C, in some instances, an internal temperature sensor 72 may be located within the optical box 30 adjacent to the fiber ferrule 33 and the diffusing lens 32 for indication of the internal optical interface temperatures.

The external and internal temperature sensors 28, 72 may be connected to the integrated electronics 16 via one or more wires 29. The integrated electronics 16 are thus configured to wirelessly transmit temperature data received from the external and internal temperature sensors 28, 72 to the CCU 10 as depicted in FIG. 1. The CCU 10 may thus be configured to control operation of the CLG 6 and/or the cooling system 9 based on this received temperature data. For example, in some instances, the CCU 10 may adjust a coolant flow rate, a pressure, and/or a temperature of water in a water chiller (e.g., water chiller 53) of the cooling system 9 based on the received temperature data to maintain the internal and/or external components of the probe device 1 at desired temperatures. For example, in some instances, the CCU 10 may be configured to ensure that the temperature of the patient tissue (e.g., as monitored by the external temperature sensors 28) is maintained below a desired tissue threshold temperature. For example, in some instances, the desired tissue threshold temperature may be 41 degrees Celsius, 45 degrees Celsius, 46 degrees Celsius, or any other threshold temperature, as desired for a given application. Similarly, in some instances, the CCU 10 may be configured to ensure that the temperature of various internal probe components (e.g., the optical window 21, the diffusing lens 32, the optical box 30) are maintained below a desired component threshold temperature (e.g., similarly monitored via the internal temperatures sensors 72).

As best shown in FIG. 3C, the coolant inlet tube 17 is arranged within an insulating sleeve 35 configured to minimize heat transfer from the ambient environment to the cooled coolant (e.g., CO₂ gas) within the coolant inlet tube 17. The coolant inlet tube 17 terminates within a coolant inlet port 38 at the optical box 30 where flowing coolant (e.g., CO2 gas) enters the optical box cavity 31 and flows onto and over an internal surface of the optical window lens 21. Further, the arrangement of the coolant inlet port 38 is configured to deposit coolant directly onto the inner surface of the optical lens to directly cool the inner surface of the optical lens 21, through which the coherent light is transmitted to the patient during use. As such, in some instances, the coolant (e.g., CO₂ gas) flows directly within the path of light transmission within the optical box 30.

Accordingly, coolant entering the optical box cavity 31 from the coolant inlet tube 17 at the inlet port 38 of the optical box 30 cools the probe distal end optics (e.g., the optical window lens 21 and the diffusing lens 32) and physical structures (e.g., the internal surfaces of the optical box cavity 31) of the optical box 30, as well as the patient's tissue contacting the optical window lens 21. That is, the coolant (e.g., CO₂ gas) flowing incident upon the inside surface of the optical window lens 21 cools the optical window lens 21 and the patient tissue in contact with the outside surface of the optical window lens 21. This cooling of the physical and optical components and interfaces at the distal end 20 of the probe device 1 improves photon transmission efficiency and overall therapeutic treatment quality. Additionally, by cooling the patient tissue, the cooling configuration may prevent patient tissue from overheating, as well as possible patient burns.

Further, as the probe device 1 is moved across the patient tissue surfaces during treatment, cooling of the patient tissue (vaginal or rectal mucosa and/or epidermal-dermal skin surface) triggers vasoconstriction that reduces blood-borne photon-absorbing chromophores, which facilitates the delivering of more photons deeper into tissue (i.e., into the lower tissue not being cooled at the surface) during the administration of photo therapy.

Accordingly, the flowing coolant (e.g., CO₂ gas) circulates through the optical box cavity 31 within the optical box 30 and is exhausted from the optical box cavity 31 of the optical box 30 via a series of passages through and around the base of the optical box 30, fiber ferrule 33, and fiber retention nut 34, as will be further discussed below. The coolant exhausted from the optical box cavity 31 of the optical box 30 is then collected or received by the collection sleeve 19 within a collection space 67.

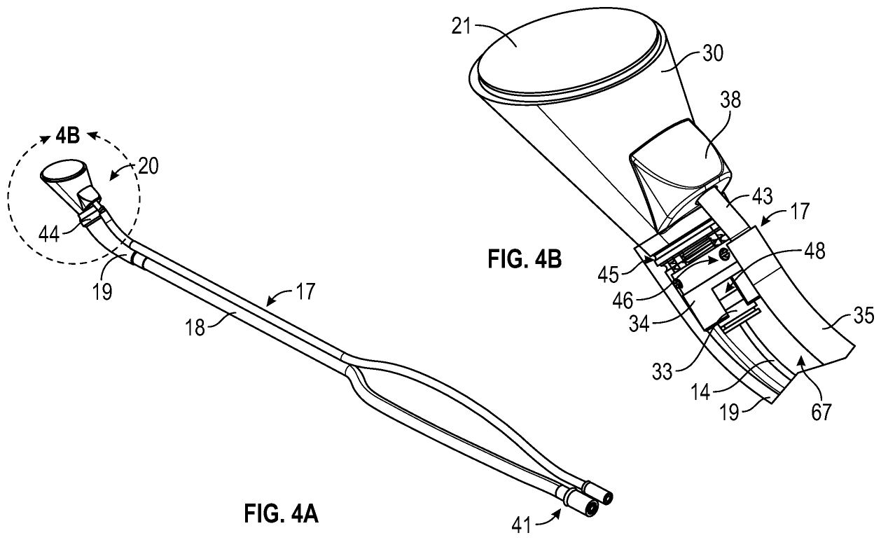

The collection sleeve 19 is located or arranged over the end of the optical box 30 and the fiber ferrule 33 with fiber retention nut 34 attached thereto, such that the one or more passages through the base of the optical box 30 (discussed further below) are fully encapsulated and all exhausted coolant is collected within the collection sleeve 19. The collection sleeve 19 is physically secured to the base of the optical box 30 by a ring 44 around the collection sleeve 19 that clamps, is crimped, or is otherwise fastened to the collection sleeve 19 and optical box 30. Additionally, an interior lip 163 of the collection sleeve 19 is configured to be received within a corresponding base groove 45 (shown in FIG. 4B) on a radially-outward facing surface of the optical box 30, which further secures the collection sleeve 19 onto the optical box 30. That is, the retention or base groove 45 locates and supports retention and sealing of the collection sleeve 19 to the optical box 30.

From the collection space 67 within the collection sleeve 19, the coolant is transferred to the coolant outlet tube 18 via a tubing junction 36 and a sealing element 37 between the collection sleeve 19 and the coolant outlet tube 18. The tubing junction 36 is integrated with and arranged within the open end of the collection sleeve 19, opposite the optical box 30. In some instances, the tubing junction 36 may be an integral and inseparable part of the collection sleeve 19. In other instances, the tubing junction 36 may alternatively be a separate component. The sealing element 37 is configured to prevent coolant from leaking between the tubing junction 36, the collection sleeve 19, and the coolant outlet tube 18 when the coolant lines are under pressure.

As illustrated, the FOC 14 extends from the end of the coolant outlet tube 18, through the tubing junction 36 and collection sleeve 19, into the fiber ferrule 33, and within the optical box 30. The stress sleeve 49 is configured to protect and support the FOC 14 beyond the end of the fiber ferrule 33 and part of the way through the length of the collection sleeve 19.

FIGS. 4A and 4B are simplified perspective and detail views of various internal components of the probe device 1. As illustrated, the distal end 20 of the probe device 1 includes the coolant inlet tube 17 feeding coolant into the optical box 30 via a coolant inlet port 38. In some instances, the coolant inlet tube 17 comprises internal tubing 43 and an external insulating sleeve 35. From the optical box 30, the coolant is then exhausted into the collection sleeve 19 and out of the coolant outlet tube 18. As alluded to above, the collection sleeve 19 is secured onto the optical box 30 via the retaining ring 44.

As best shown in FIG. 4B, the fiber ferrule 33 with the FOC 14 is located and retained within the optical box 30 by the fiber retention nut 34. The fiber retention nut 34 is secured onto the optical box 30 by a plurality of set screws 46. It should be appreciated that the fiber retention nut 34 could be secured in a variety of other suitable methods.

As referenced above, the collection sleeve 19 installed on optical box 30 results in a coolant collection space 67 through which the fiber optic cable 14 passes and exhausted coolant (e.g., CO₂ gas) is directed into the coolant outlet tube 18. Specifically, a plurality of openings 48 in the fiber retention nut 34 allow the coolant (e.g., CO₂ gas) exiting the optical box 30 to flow freely into the collection sleeve 19, as will be described in detail below.

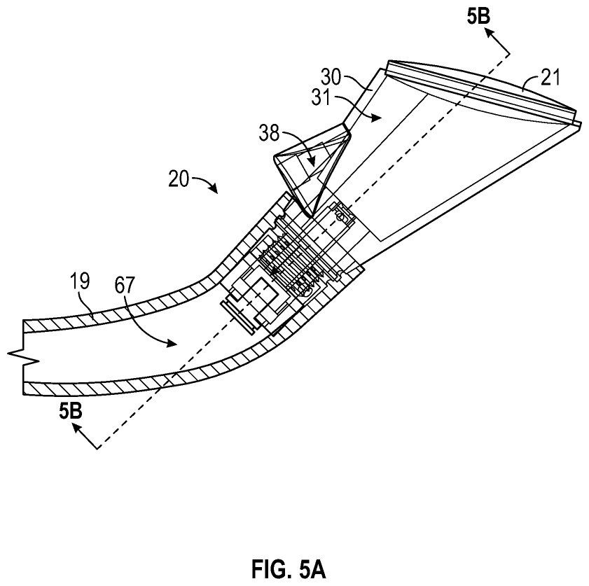

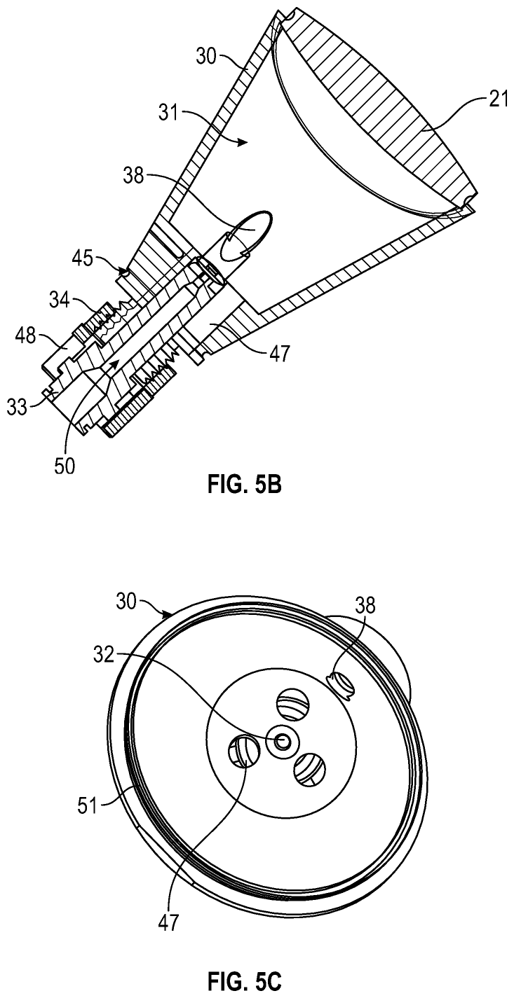

FIGS. 5A-5C are various views illustrating cooling flow paths and details of the probe device 1. As referenced above, the distal end 20 of the probe device 1 comprises the optical box 30 and the optical window lens 21, between which is the optical box cavity 31 into which flows cooled coolant (e.g., CO₂ gas) from a closed loop cooling system (e.g., cooling system 9) enters via inlet port 38 and exits into the coolant collection space 67 formed by the collection sleeve 19 at the base of the optical box 30. As best shown in FIGS. 5B and 5C, the optical box 30 has a plurality of vent ports 47 located radially around the diffusing lens 32. The vent ports 47 extend through the end of the optical box 30 and interface with openings 48 in the fiber retention nut 34, thereby allowing smooth uninhibited coolant flow and cooling from the optical box 30 along the external surfaces of the fiber ferrule 33 and into the collection sleeve 19. Accordingly, the coolant enters the optical box 30 through the inlet port 38 and exits through the vent ports 47.

The optical box 30 includes a lens seat 51 at the open end of the optical box 30 opposite the fiber ferrule 33 and diffusing lens 32. The lens seat 51 is configured to receive the optical window lens 21 when the probe device 1 is assembled. In some instances, the optical window lens 21 may be held within the lens seat 51 by the end cap 27. In other instances, the optical window lens 21 may be held within the lens seat 51 by other suitable methods, such as, for example, adhesives or fasteners.

As illustrated, the fiber ferrules 33 includes a fiber bore 50. The fiber bore 50 is configured to receive and retain the FOC 14 when the probe device 1 is assembled. In some instances, the FOC 14 may be retained within the fiber bore 50 via an adhesive (e.g., epoxy), fasteners (e.g., set screws), or any other suitable retention method, as desired for a given application.

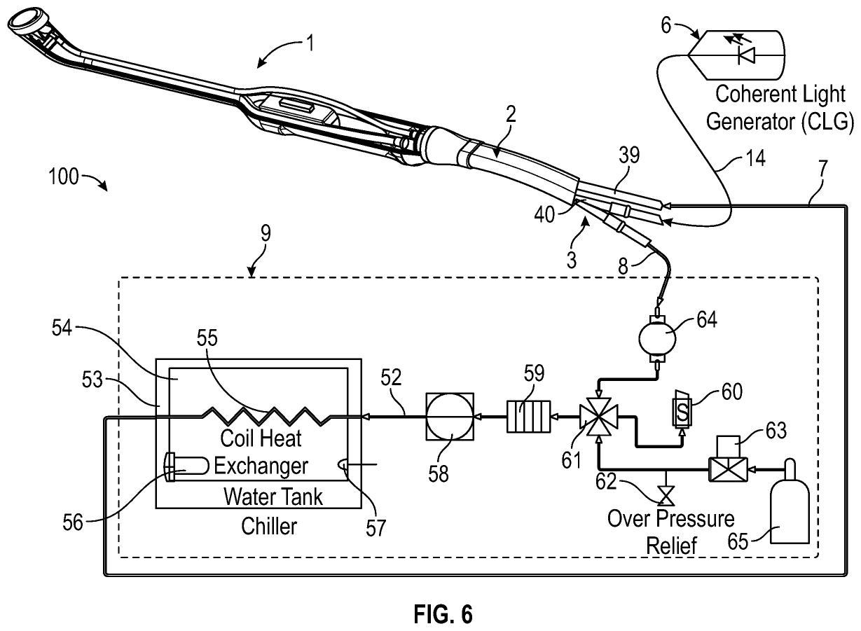

FIG. 6 is a schematic representation of the phototherapy system 100, showing various components of the cooling system 9, according to an exemplary embodiment. As referenced above, the cooling system 9 is configured to provide chilled coolant to the probe device 1 via cooling system outlet tube 7, which is connected to the coolant inlet tube 39 of the umbilical cable 2. The cooling system 9 is further configured to receive the heated (e.g., via the cooling of the various optical components of the probe device 1) coolant via the cooling system inlet tube 8, which receives the coolant via the coolant outlet tube 40 of the umbilical cable 2.

In some instances, the cooling system 9 is a closed-loop cooling circuit configured to recirculate the same coolant throughout the system. The cooling system 9 may include a variety of coolant flow, temperature, and pressure monitoring and control elements including, but not limited to, a coolant flow pump 64, a four (4) way valve 61, a purge valve 60, a coolant tank 65 (e.g., a bone dry CO₂ gas tank), a tank outlet pressure regulator 63, an overpressure relief valve 62, a filter-desiccator 59, a mass flow meter 58, and a water chiller 53.

The purge valve 60 is configured to allow the cooling system 9 to be de-pressurized when the 4-way valve 61 is in the appropriate position and the purge valve 60 is actuated. In some instances, the purge valve 60 may be manually or electronically actuated. In some instances, the purge valve 60 may be implemented with or without a silencing feature to muffle the sound of exhausting coolant (e.g., CO₂ gas).

Coolant (e.g., CO₂ gas) enters the closed-loop cooling system 9 from the coolant tank 65 during a process referred to as “charging” when the 4-way valve 61 is in the appropriate position. The overpressure relief valve 62 automatically prevents over-pressurizing during the charging process. That is, the overpressure relief valve 62 is configured to provide automatic pressure relief to prevent over pressurizing the closed-loop cooling system 9.

In some instances, the filter-desiccator 59 is a single unit which accomplishes both filtering of the coolant (e.g., CO₂ gas) for physical and biological contaminants in the closed-loop coolant system, as well as drying of the coolant (e.g., a gas) during normal use. In some other instances, the filter-desiccator 59 is a combination of elements which accomplish these functions.

The mass flow meter 58 is an electronic monitoring device which provides information to the CCU 10 regarding the flow rate, pressure, and temperature of the flowing coolant in the closed-loop cooling system 9. In some instances, data from the mass flow meter 58 drives the CCU 10 to make changes to the operational settings of the water chiller 53 and the pump 64 to maintain the system at designated flow, pressure, and temperature levels.

In some instances, the water chiller 53 includes an integrated water tank 54, a cooling module 56, and a temperature sensor 57. The integrated water tank 54 has a coil heat exchanger 55 configured to allow heat transfer between the water tank 54 and the coolant flowing through the coil heat exchanger 55 within the closed-loop cooling system 9. The cooling module 56 is configured to circulate and cool the water within the water tank 54 of the water chiller 53. The temperature sensor 57 is configured to monitor the water temperature in the water tank 54 and provides water temperature data to the CCU 10 to be used in controlling the cooling system 9. For example, the CCU 10 may be used to selectively raise or lower the temperature of the water in the water tank 54 (e.g., based on temperature data received from the external and internal temperature sensors 28, 72). Further, in some instances, the water in the water tank 54 may be mixed with another liquid to allow for sub-zero (Celsius) temperatures (e.g., temperatures below the freezing temperature of water). For example, in some instances, the water may be mixed with methanol alcohol to allow for the sub-zero (Celsius) temperatures.

Accordingly, under normal operating conditions, the pump 64 pushes the coolant (e.g., CO₂ gas) through the closed-loop cooling system 9, through the 4-way valve 61, the filter-desiccator 59, the mass flow meter 58, the tubing 52 within the cooling system 9, and into the coil heat exchanger 55 submerged in the water tank 54 of the water chiller 53, where the coolant is cooled. The coolant then travels through cooling system outlet tube 7 to the umbilical cable 2 and the probe device 1, where the coolant is used to cool various components of the probe device 1 (e.g., the distal end 20 and patient tissue interfaces). After cooling the various components of the probe device 1, the coolant carries heat generated during photobiomodulation therapy back to the cooling system 9 to be dissipated and re-circulated for continuous cooling.

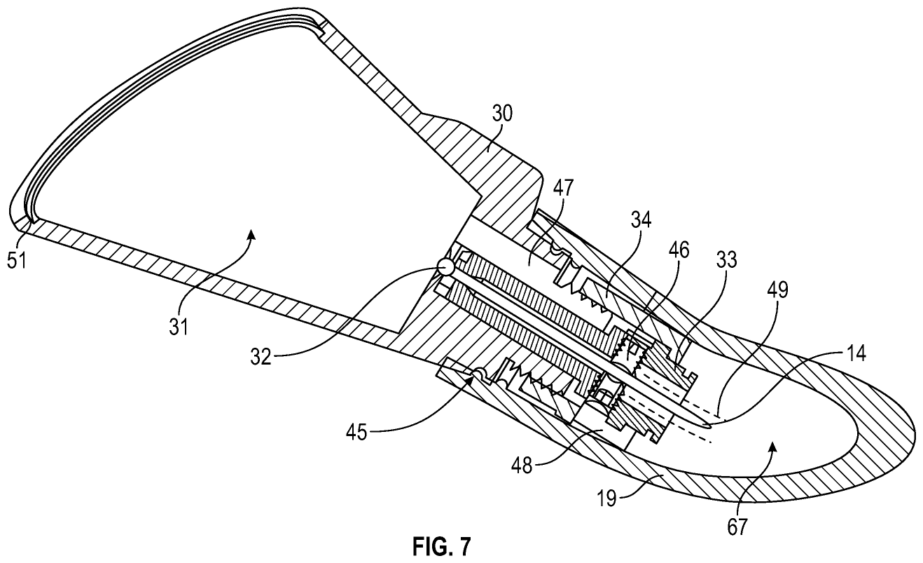

FIG. 7 is a section view of the distal end of the probe device, illustrating an alternate embodiment of a fiber retention mechanism within the probe. For example, as illustrated, in some instances, a plurality of set screws 46 located within the fiber ferrule 33 may be used to mechanically secure the FOC 14 and/or stress sleeve 49 within the fiber ferrule 33. In some instances, four set screws 46 may be used. In other instances, more or less than four set screws 46 may be used. Similarly, in these instances, the optical box cavity 31 of the optical box 30 may still include vent ports 47 and the fiber retention nut 34 may include openings 48 configured to allow flowing coolant to exhaust from the optical box 30 into the collection sleeve 19, where the coolant is collected in collection space 67.

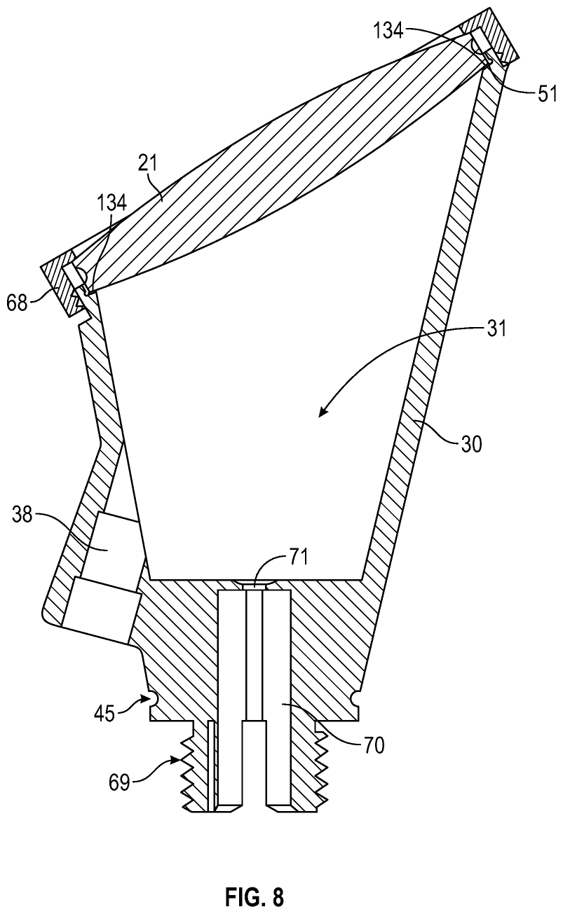

FIG. 8 is a section view of the distal end of the probe device 1 including an alternative optical window lens retention scheme. Specifically, the optical window lens 21 of the optical box 30 is similarly located on a lens seat 51. However, the optical window lens 21 is retained to the optical box, within the lens seat 51, by a retaining ring 68. In some instances, the retaining ring 68 may be threaded onto the optical box 30. In other instances, the retaining ring 68 may be retained on the optical box 30 via various other methods, such as, for example, adhesives, a snap-fit connection, or any other suitable method.

In the illustrated embodiment shown in FIG. 8, the retaining ring 68 captures the edges of the optical window lens 21 and is secured to an external thread on the optical box 30. In some other embodiments, the retaining ring 68 may be threaded into an internal optical box thread. Further, in some embodiments, the retaining ring 68 could be configured to contact a tapered external surface of the optical window lens 21 instead of the top surface, as shown in FIG. 8. In some instances, the retaining ring 68 may compress the optical window lens 21 against the lens seat 51 to ensure that a seal is created between the optical window lens 21 and the lens seat 51 to prevent coolant flowing within the optical box cavity 31 from leaking out. In some instances, as shown in FIG. 8, a compliant gasket 134 may further be included between the optical window lens 21 and the lens seat 51 to further improve the seal formed between the optical window lens 21 and the lens seat 51.

Additionally, a fiber ferrule bore 70 may be configured to locate or arrange the fiber ferrule 33 with the diffusing lens 32 being arranged within a seat 71 at the distal end of the fiber ferrule bore 70. The base of the optical box 30 may further include external threads 69 configured to interface with the fiber retention nut 34.

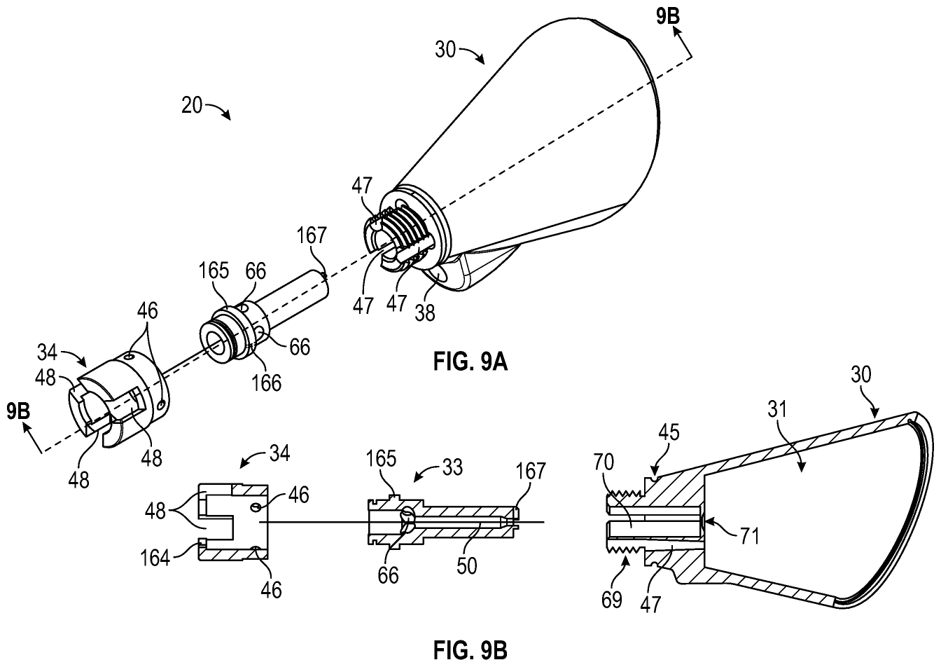

FIGS. 9A and 9B are exploded perspective and sectional views of the distal end 20 of the probe device 1, illustrating the optical box 30, the fiber ferrule 33, and the fiber retention nut 34 and the various coolant cooling paths and interfaces. As discussed above, the optical box 30 includes the coolant inlet port 38 and the plurality of vent ports 47. The fiber ferrule 33 includes a plurality of fiber retention screw holes 66 configured to receive a plurality of corresponding fiber retention set screws. The fiber retention nut 34 similarly includes a plurality of fiber retention screw holes 66 configured to receive a plurality of corresponding holding screws and a plurality of vent openings 48.

During operation, cooled coolant enters the optical box 30 at inlet port 38 and circulates through the optical box cavity 31, cooling various components of the optical box 30, as well as the patient's tissues, as described in detail throughout the present disclosure. The coolant is then exhausted from the optical box 30 via the plurality of vent ports 47, shown as 3 equally-spaced holes around the central axis of the fiber ferrule bore 70 in the illustrative example provided in FIG. 9A. The vent ports 47 are located and sized such that they partially break through (i.e., create an opening for coolant to flow through) to both the internal bore of the fiber ferrule bore 70 and the external threads 69 configured to engage the fiber retention nut 34.

Accordingly, exhausted coolant from the optical box 30 flows through the vent ports 47 around the external surfaces of the fiber ferrule 33 when installed into the fiber ferrule bore 70 of the optical box 30. Exhausted coolant flowing through the vent ports 47 is then free to flow into and through the fiber retention nut 34 by way of the vent ports 47 which break through the external threads 69, thereby creating an opening (i.e., a fluidly-connected channel) between the vent ports 47 and the vent openings 48 of the fiber retention nut 34.

Specifically, with the fiber ferrule 33 slid into the fiber ferrule bore 70, the fiber retention nut 34 is slid over the fiber ferrule 33 and coupled by threads to the optical box 30 via the external threads 69. As the fiber retention nut 34 is slid over the fiber ferrule 33, a ferrule engaging lip 164 of the fiber retention nut 34 is configured to contact a retention nut engaging ridge 165, thereby retaining the fiber ferrule 33 within the fiber ferrule bore 70. However, when the ferrule engaging lip 164 contacts the retention nut engaging ridge 165, the vent openings 48 are configured to extend beyond the retention nut engaging ridge 165 (as best shown in FIG. 5B), thereby creating the opening between the vent ports 47 and the vent openings 48. Accordingly, the vent ports 47 and the vent openings 48 result in a continuous, unobstructed flow path between the optical box cavity 31 and the collection sleeve 19, allowing the coolant (e.g., the CO₂ gas) to flow through and cool the various optic components of the probe device 1.

Further, in some instances, the retention nut engaging ridge 165 may optionally include one or more axial channels 166 (shown by dashed lines in FIG. 9A) to improve the coolant flow rate within the probe device 1. For example, although one axial channel 166 is shown, in some instances, three axial channels 166 may be provided through the retention nut engaging ridge 165 (which may substantially align with the vent openings 48). In some instances, the axial channels 166 may be approximately half the width of the vent openings 48.

The fiber ferrule 33 further includes a winged tip 167 atop which the diffusing lens 32 (e.g., the ball lens) is configured to sit when the fiber ferrule 33 is slide into the fiber ferrule bore 70. The winged tip 167 comprises a pair of aligned linear protrusions extending axially away from an axial end of the fiber ferrule 33 on either side of the fiber bore 50. The winged tip 167 allows for coolant that flows through the vent ports 47 to swirl around and directly cool the diffusing lens 32 on either side of the winged tip 167. It should be appreciated that the winged tip 167 could be sized or shaped differently, as desired for a given application.

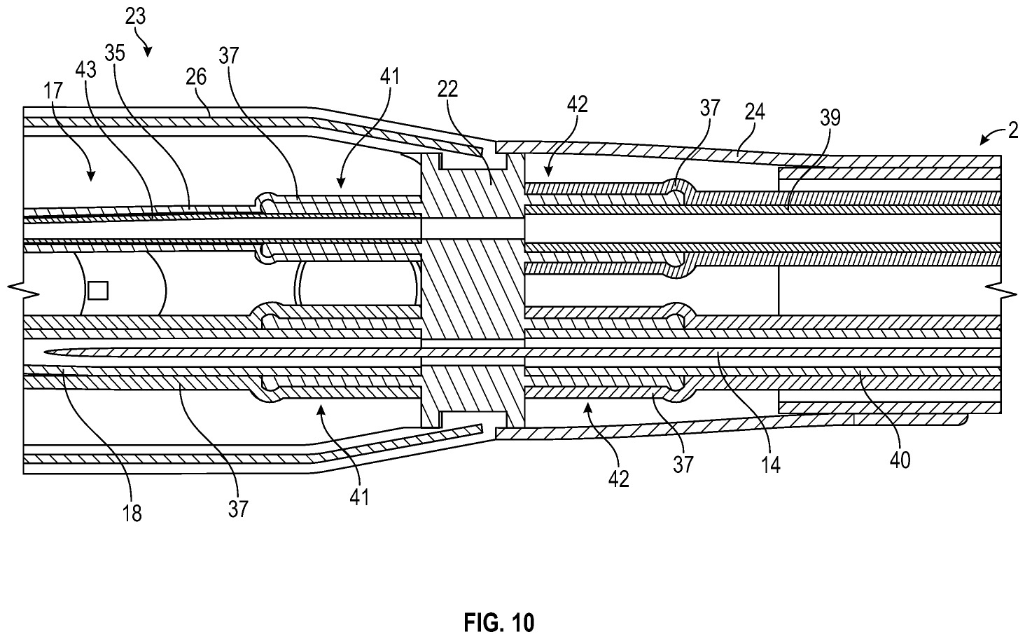

Referring now to FIG. 10, a section view of a junction portion of the probe device 1 is shown, depicting the interface between the probe device 1 and the umbilical cable 2 with various tubing connections between the umbilical cable 2 and probe device 1, as well as the FOC 14, within the tubing junction 22 at the proximal end of the probe device 1. For example, the proximal end 23 of the probe device 1 includes the tubing junction 22 between the probe enclosure (the bottom cover 26 being shown in FIG. 10) and the strain relief collar 24 (and the umbilical cable 2).

For example, the tubing junction 22 provides an interface between the probe internal tube end connections 41 and the probe external tube end connections 42. In some instances, the external tube end connections 42 for receiving the coolant inlet tube 39 from the umbilical cable 2 is installed within/onto the tubing junction 22 and secured/sealed via a sealing element 37. In the illustrative example provided in FIG. 10, the sealing element 37 is a heat-shrink-type element. However, in other instances, the sealing element 37 may be various other types of sealing elements (e.g., epoxy).

The internal tube end connection 41 connected to the internal inlet coolant tube 17 may comprise the internal tubing 43, through which the coolant flows, installed within/onto tubing junction 22 and an insulating sleeve 35. In some instances, as shown in the illustrative example provided in FIG. 10, the insulating sleeve 35 (e.g., a portion thereof) may also function as the sealing element 37 between the internal tubing 43 and tubing junction 22.

The internal tube end connection 41 for the internal coolant outlet tube 18 is installed within/onto the tubing junction 22 and secured/sealed via sealing element 37. The external tube end connections 42 for coolant outlet tube 40 within the umbilical cable 2 is installed within/onto the tubing junction 22 and secured/sealed via sealing element 37. Additionally, the FOC 14 is arranged within the external coolant outlet tube 40 and internal coolant outlet tube 18, thereby allowing a continuous, unobstructed FOC 14 between the coherent light generator 6 and the optical box 30 for the delivery of therapeutic light energy to the patient. It should be understood that, although the FOC 14 is shown terminating within the internal coolant outlet tube 18, as assembled, the FOC 14 extends to the optical box 30, as described herein.

In some instances, insulation is included on the various components of the cooling system 82 (as well as the cooling system 9 shown in FIG. 1). For example, in some instances, insulation is used to encase or otherwise envelop all or some of the various tubing and other components having coolant flowing therethrough within the cooling system 82 (or the cooling system 9) to reduce undesired heat from entering the cooling system 82 (or the cooling system 9). For example, in some instances, various insulating sleeves (e.g., similar to the insulating sleeve 35) may be utilized on any/all coolant tubing within the cooling systems described herein. Similar insulating sleeves may be sized and configured to envelop the various other components having coolant flowing therethrough, as desired for a given application. In some instances, the insulating sleeve 35 and/or the various other insulation within the cooling systems described herein may comprise rubber, potting, plastic, or other materials. In some instances, some or all of the insulation may be provided via an air gap or a vacuum-sealed gap between components and the ambient environment. Further, in some instances, the various air gaps and cavities within the probe device 1 (or the probe device 80) may be filled in with insulation (e.g., foam, rubber, etc.) to keep internally generated heat within the probe device from heating the patient tissue. In some instances, filling in the air gaps and cavities within the probe device may similarly prevent heat from the patient from artificially heating the internal components of the probe device.

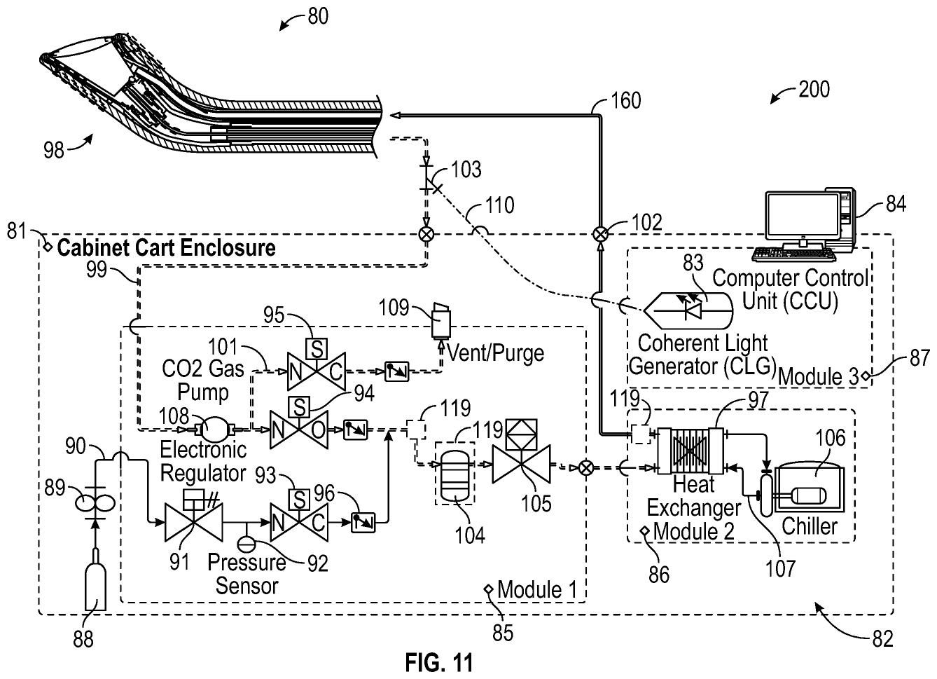

FIG. 11 is a schematic representation of another phototherapy system 200, according to an exemplary embodiment. It should be appreciated that any of the components of either of the system 100 or the system 200 may be used in place of or in addition to the various components of the system 100 or the system 200 without departing from the scope of the present disclosure.

For example, the phototherapy system 200 similarly includes a probe device 80 for the application of PBMT, a cooling system 82, a coherent light generator (CLG) 83, and a computer control unit (CCU 84). In some instances, the probe device 80 is similarly a handheld probe device. The phototherapy system 200 further includes an enclosure 81 (e.g., a cabinet, cart, or other type of enclosure) having one or more integrated system modules configured to encompass and/or hold the cooling system 82, the coherent light generator 83, and various electronics including the CCU 84 for the operation of the probe device 80.

In FIG. 11, the cooling system 82 comprises the collection of components co-located within a first integrated module 85 and a second integrated module 86. The CLG 83 and CCU 84, in addition to various other control electronics are combined within a third integrated module 87. For example, these modules may be various areas or sections within the enclosure 81. It should be appreciated that, in some instances, various other configurations are possible, and various components described herein may be provided within the same or different modules.

Prior to operation, the cooling system 82 is supplied (e.g., charged) from a source supply tank 88 (e.g., a CO₂ gas supply tank). In some instances, the supply tank 88 may be mounted within the enclosure 81. Additionally, in some instances, the supply tank 88 may be a small refillable tank configured to store medical-grade bone-dry or similarly clean, dry CO₂ gas. In some other instances, various other types of coolant may be utilized. The supply tank 88 may have a pressure, when full, of between 400 psi and 1,200 psi. In some instances, the coolant may be a different gas, a mixture of gasses, a liquid, or a mixture of liquids as desired for a given application. In these instances, the gas, mixture of gasses, liquid, or mixture of liquids utilized may be selected based on a suitability of the coolant for use with PBMT. This suitability may be based on an amount of photon energy the coolant would absorb (e.g., does the gas or mixture absorb large amounts of photon energy) and whether the coolant is deemed safe to a patient, both internally and externally. As described herein, in some embodiments, the systems 100, 200 are configured to utilize bone-dry, clean, medical-grade CO₂ gas. In some other embodiments, the systems 100, 200 are configured to utilize chilled air gas, chilled nitrogen gas, chilled water, or any of a variety of other coolants, as desired for a given application.

A tank regulator 89 is configured to restrict the supply of coolant from the supply tank 88 to the cooling system 82 by limiting the pressure of the coolant which is allowed to enter the cooling system 82 at the first integrated module 85. In some instances, the tank regulator 89 is configured to bring the pressure down to approximately 100 psi.

Accordingly, coolant (e.g., CO₂ gas) slowly flows from the tank regulator 89 into an inlet path 90 of the cooling system 82 (depicted in FIG. 11 by a solid line with flow in the direction indicated by the arrows on the line). An electronic pressure regulator 91 further controls the coolant pressure within the inlet path 90 to a desired target operating pressure for the cooling system 82. The electronic pressure regulator 91 may be connected to and controlled by the CCU 84. In some instances, operating pressure for the cooling system 82, as controlled by the electronic pressure regulator 91, may be between 20 psi and 35 psi. However, in some instances, the operating pressure could range between 10 psi and 100 psi.

A pressure sensor 92 downstream from the electronic pressure regulator 91 within the inlet path 90 of the cooling system 82 provides precise pressure feedback to the CCU 84 representing the regulated pressure intended for introduction into the cooling system 82. The cooling system 82 further includes one or more solenoid valves 93, 94 and 95 (depicted in FIG. 11 with an “S” indicating solenoid). Solenoid valve 93 and solenoid valve 95 are normally closed valves (indicated by the “N” and “C” within FIG. 11). The solenoid valve 93 shown in the inlet path 90 is controlled by the CCU 84, and is opened as-needed to charge the cooling system 82 (e.g., by adding CO2 gas to reach a desired pressure). Solenoid valve 94 is a normally open valve (as indicated by the “N” and “O” within FIG. 11). The cooling system 82 may further include one or more check valves 96, which may be utilized to control the flow of coolant through the cooling system 82 (e.g., by blocking flow in one direction only allowing coolant to flow in the direction indicated by the arrows within the check valve 96 illustrations).

The main coolant flow loop of the cooling system 82 is a closed loop circuit during normal operation, with the coolant circulating through the probe device 80 to provide cooling during the delivery of PBMT. A heat exchanger 97 is located within the main coolant flow loop to cool the coolant and remove heat carried away from distal end 98 of the probe device 80, where the PBMT is applied.

There are two sides to the main coolant flow loop. That is, the main coolant flow loop includes a hot side 99, containing heated coolant flowing from the probe device 80 to the heat exchanger 97, and cold side 160, containing cooled coolant flowing from the heat exchanger 97 to the probe device 80. The hot side 99 is depicted in FIG. 11 by a dashed double line “==” and comprises tubing between the depicted components and any needed connectors and adapters required to facilitate assembly. For example, in some instances, the connectors used may be dry-break quick disconnect pneumatic connectors. In some other instances, the connectors used may be other types of connectors, as necessary for differing types of coolants used (e.g., liquid coolants). The cold side 160 is depicted by a solid double line “===” and comprises tubing between the depicted components and any needed connectors and adapters required to facilitate assembly. For example, in some instances, the connectors used may be dry-break quick disconnect pneumatic connectors. In some other instances, the connectors used may be other types of connectors, as necessary for differing types of coolants used (e.g., liquid coolants). Similarly the inlet path 90 and a vent path 101 (leading to a vent 109) utilize tubing or other functional structure to serve as a flow path between components and other tubes. In some instances, the tubing used within the cooling system 82 may be plastic or stainless steel. In other instances, the tubing could be any type of hollow or tubular structure through which a fluid (e.g., a gas or liquid) can flow without leaks.

Additionally, in some instances, various connectors and adapters used throughout the cooling system 82 may be standard fluidics and pneumatics components. In other instances, the various connectors and adapters may be custom interfaces for joining sections of similar or different tubing, as well as connecting that tubing to other system components. For example, in some instances, the cooling system 82 includes a variety of connectors and adapters, including, but not limited to, threaded connector-to-barb tube fittings, threaded connector adapters for different threads, dry-break quick disconnect pneumatic connectors, other fluidic connectors, and junction components with two or more inlets and/or outlets for joining or separating various flow paths.

In some instances, the cooling system 82 may further include one or more quick connection fittings 102. The quick connection fittings 102 are connectors having mating portions that allow for connection and removal without tools. In some instances, the quick connection fittings 102 also have sealing functions that prevent or minimize losses of pressure and contamination of the cooling system 82. The cooling system 82 may additionally include a “T” or wye fitting 103 with 3 or more ports, similar to the wye junction 3 discussed above.

Accordingly, prior to operation, coolant from the supply tank 88 is allowed to enter the closed loop recirculating portion of the cooling system 82 when the normally closed solenoid valve 93 opens to “charge” the cooling system 82. Then, during operation, coolant from the inlet path 90 flows into the hot side 99 adjacent to a check valve 96 to force inflow to enter the loop in one direction shown by the arrows. The coolant entering the closed loop flow path flows towards the heat exchanger 97, passing through a filter and/or desiccation device 104 to clean and dry the coolant (e.g., CO2 gas) and a mass flow meter and/or other sensing devices 105.

In some instances, the filter and/or desiccation device 104 may be an all-in-one device configured to perform both filtering and desiccation in one operation. In some other instances, the cooling system 82 may include separate, independent filtering and desiccation devices connected to each other in series (usually with filtering first). Further, in some instances, the filter and desiccation device 104 may be replaced with a liquid filter device in the scenario where a liquid coolant is utilized. In some instances, additional quick connection fittings 102 may be used on either side of the filter and/or desiccation device 104 or other component(s) to facilitate tool-less service and replacements. Additionally, in some instances, the device(s) which are utilized to accomplish filtering and/or desiccation may have replaceable consumables for the filter and/or desiccation media.

In some instances, the cooling system 82 further includes one or more sterilization components 119. As illustrated in FIG. 11, in some instances, one or more sterilization components 119 may be arranged upstream of the filter and/or desiccation device 104. Additionally, in some instances, one or more sterilization components 119 may be arranged downstream of the heat exchanger 97. However, it should be appreciated that, in other instances, the sterilization components 119 may be arranged anywhere within the cooling system 82, as desired for a given application.

The sterilization components 119 may be anti-viral, anti-bacterial, antimicrobial, and/or any other type of sterilization components. In some instances, the sterilization components 119 are ultraviolet (UV) light treatment components configured to apply ultraviolet (UV) light to the coolant within the cooling lines to restrict mold and/or bacterial growth within the cooling system 82. For example, one or more of the sterilization components 119 may be an inline UV light sterilization unit or an external-to-the-tubing UV light sterilization unit. In either case, the UV sterilization unit may be configured to keep the internal environment of the cooling system 82 (e.g., the coolant flowing within the cooling system 82) sterile or close to sterile (e.g., bacteriostatic and/or bactericidal). In some instances, the cooling system 82 may additionally or alternatively include a sterilization component 119 comprising an external UV light unit enveloping and illuminating the coolant within the filter and/or desiccation device 104 and/or illuminating an intake channel (e.g., intake tubing) of the filter and/or desiccation device 104.

In some instances, in addition to or in place of the sterilization components 119, the cooling system 82 includes antimicrobial tubing (e.g., antimicrobial impregnated plastic tubing) and bacterial-resistant fittings (e.g., bacterial-resistant stainless steel fittings). In these instances, the antimicrobial tubing and bacterial-resistant fittings are configured to reduce potential bacterial and/or viral particle build up within the cooling system 82, which has a warm to cold circulating coolant internal environment.

The mass flow meter and/or sensing devices 105 provides various information about the coolant flowing through the cooling system 82 to the CCU 84. In some instances, the sensing device 105 may be an all-encompassing sensing device capable of measuring mass flow of the coolant, the system pressure, and the coolant temperature. In some other instances, separate devices may be used to capture all necessary information. Additionally, in some instances, additional information like relative humidity could be measured and utilized (e.g., in the scenario where a gaseous coolant is utilized).

In some instances, the heat exchanger 97 may be a cross-flow type plate heat exchanger coupled on one side with the coolant flow path and on the other with a chilled heat transfer fluid flowing through a chiller 106. The chiller 106 is configured to use the heat transfer fluid to remove heat from the coolant as it flows through the heat exchanger 97 and to cool the heat transfer fluid again as it flows back through the chiller 106. The chiller 106 may be configured to use thermoelectric elements or other cooling methods to remove heat from the heat transfer fluid flowing though tubing 107 (depicted in FIG. 11 as a dot-dash line “.-.-”) between the chiller 106 and the heat exchanger 97. In some instances, the chiller 106 may use water, methanol, propylene glycol, or any other suitable liquid or a mixture of fluids as the heat transfer medium. The chiller 106 further includes an integrated pump configured to circulate the heat transfer fluid through the chiller 106 and to/from the heat exchanger 97 through tubing 107. The chiller 106 includes a variety of sensors that are connected to the CCU 84, which may be configured to set the temperature of the chiller 106 and/or monitor and control various other aspects of the chiller 106. In some instances, the CCU 84 may be configured to actively adjust the temperature of the chiller 106 during operation to allow for a variety of cooling capabilities at differing coolant flow rates.

After the coolant has passed through the heat exchanger, the flowing coolant leaving the heat exchanger 97 on the cold side 160 flows to the distal end 98 of the probe device 80 to directly cool the internal and external optical interfaces and other components. From the probe device 80, the coolant flows toward a coolant pump 108 located on the hot side 99, downstream from the probe device 80. The coolant pump 108 is configured to simultaneously push coolant from the pump 108, through the heat exchanger 97 and ultimately to the probe device 80, and to pull coolant out of the probe device 80 through the exhaust channels towards the pump 108. In some instances, the coolant pump 108 may be a diaphragm type pump. In some other instances, the pump 108 may be a piston pump, a centrifugal pump, a peristaltic pump, or any other suitable pump type. In any case, the pump 108 may be configured to ensure that the system pressure is equal on both sides of the pump 108 at steady state with minimal pressure drop during normal operation.

Arranged downstream from the coolant pump 108 is the normally open solenoid valve 94 immediately downstream of a junction for a vent path 101 (depicted using double dotted “: : : : : ” lines in FIG. 11). The normally open solenoid valve 94 may be used (e.g., selectively closed) during specific purging and filling routines to close the flow loop and direct flow out of the cooling system 82 via the vent 109.

In some instances, the vent path 101 may have a normally-closed solenoid valve 95, followed by a check valve 96 and the vent 109. The normally-closed solenoid valve 95 in the vent path 101 may be used (e.g., selectively opened) to exhaust the coolant from the cooling system 82 (e.g., to lower system pressure, perform maintenance, or during a purging routine to push ambient air out of the cooling system 82). The check valve 96 in the vent path 101 is oriented such that flow can only go towards the vent 109, thereby preventing ambient air from being drawn into the cooling system 82 through the vent path 101. The vent 109 may be a simple exhaust. For example, the vent 109 may comprise an opening, a silencer, or a baffled outlet with or without its own filter or other sound dampening mechanisms.

As illustrated in FIG. 11, the CLG 83 is similarly configured to emit the therapeutic light energy through a fiber optic cable 110, which similarly enters the hot side 99 tubing via the 3-way wye junction 103, where it is then routed to the distal end 98 of the probe device 80 for the delivery PBMT, in a similar manner to that described above, with respect to the phototherapy system 100.

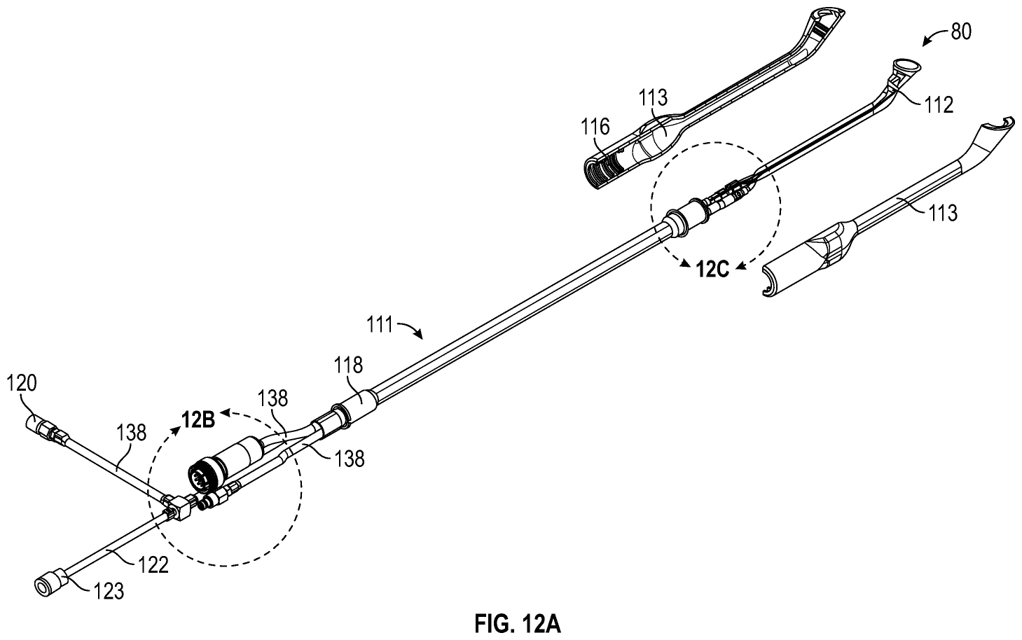

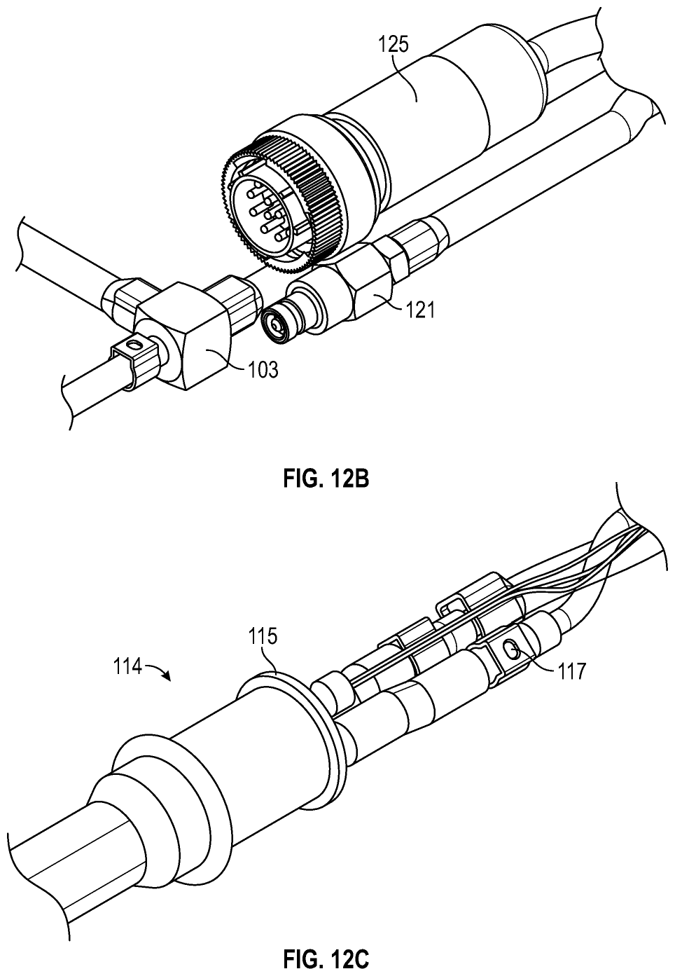

Referring now to FIGS. 12A-12C, a partially-exploded view and various detail views of the probe device 80 are shown. As depicted, in some instances, the probe device 80 may be a hardwired probe that does not include wireless electronics. However, it should be appreciated that the probe device 80 may alternatively incorporate similar wireless features to those discussed above, with respect to the probe device 1.

The probe device 80 shown in FIGS. 12A-12C utilizes direct hardwire connections for probe sensing devices (e.g., thermocouples) to the CCU 84 via an umbilical cable 111. That is, the probe device 80 shown in FIGS. 12A-12C does not include integrated circuits, battery, motion sensing devices, or wireless communication component generally for wirelessly communicating between the probe device 80 and CCU 84.

The internal components 112 of the probe device 80 are enclosed within a body 113 comprising two or more pieces. As shown in FIG. 12A, the body 113 comprises two half body parts, which may be referred to as a clamshell. The enclosing body 113 is configured to hold and locate or otherwise arrange the internal components 112 of the probe device 80, and serves as a protective barrier between the external environment and the internal components 112.

As best shown in FIG. 12C, a junction assembly 114 at a proximal end of the probe device 80 is configured to provide a robust interface between the body 113 of the probe device 80 and the umbilical cable 111. Various physical features of the junction assembly 114 are configured to interface with various corresponding physical features of the body 113. For example, a rib feature 115 of the junction assembly 114 is configured to interface with a groove 116 of the body 113 when assembled. The junction assembly 114 comprises a multitude of layers of tubes, sleeves, heat shrinks, adhesives, collars, and/or other ridged supports configured to securely retain and support all connections during use of the probe device 80 (e.g., similar to the tubing junction 22 discussed above). For example, in some instances, various crimp-on clamps 117 or other retention device(s) may be used in addition to fittings like barbed tube connections to securely retain tubing and prevent leaking.

Referring again to FIG. 12A, an umbilical interface 118 on the proximal end of the umbilical cable 111 may provide similar integrity and durability to connections and exposed tubing 138 extending between the umbilical cable 111 and various connections to the cooling system 82, the coherent light generator 83, the CCU 84, and/or various other connected electronic systems (e.g., a data acquisition system).

The proximal end components of the umbilical cable 111 (communicably coupled to the probe device 80) include the exposed tubing 138, a hot-side cooling system connection 120, and a cold-side cooling system connection 121. In some instances, the hot-side cooling system connection 120 and/or the cold-side cooling system connection 121 may be quick-connect fittings (e.g., the quick connection fitting 102 shown in FIG. 11).

The proximal end components further include a fiber optic cable tube 122, an SMA connector or CLG connector 123, the T-fitting 103, and a multi-pin connector 125. The fiber optic cable tube 122 and SMA connector or CLG connector 123 allow for connection between the probe device 80 and the CLG 83 via the FOC 110. The T-fitting 103 is configured to allow for the FOC 110 within the fiber optic cable tube 122 to enter the exposed tubing 138 of the hot side 99. The multi-pin connector 125 is configured to allow for thermocouples and/or other hardwired electronics within the probe device 80 to connect to the CCU 84.

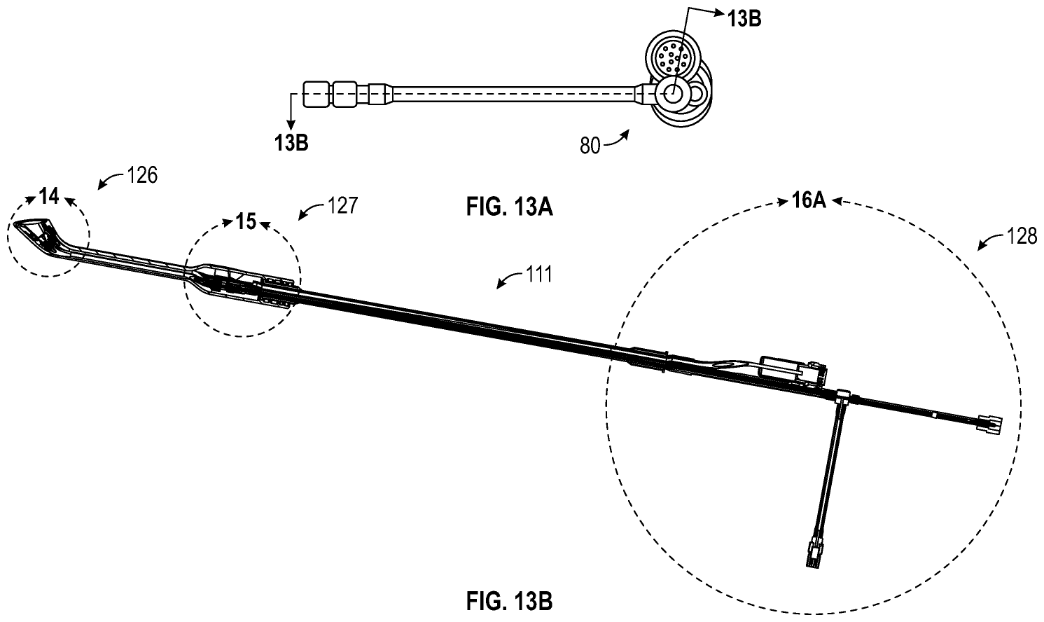

FIG. 13A-16C are various views of the probe device 80 and the proximal end components of the umbilical cable 111 showing various internal characteristics. As shown in FIGS. 13A and 13B, the probe device 80 includes a distal end 126 and a handle section 127, and the umbilical cable 111 includes a proximal end 128. Similar to the umbilical cable 2 discussed above, the umbilical cable 111 may be a flexible section of conduit with one or more channels through which tubing and wires pass that connects the handle section 127 of the probe device 80 to the connections at the proximal end 128 of the umbilical cable 111.

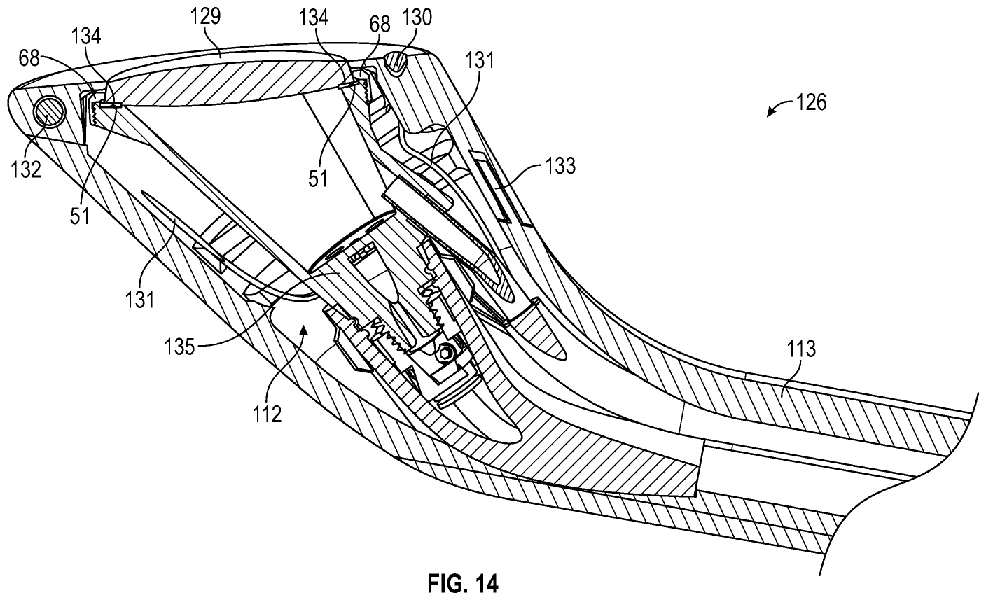

With specific reference to FIG. 14, a detailed section view of the distal end 126 of the probe device 80 taken at an off-center plane (i.e., not through a center of the distal end) is shown. As illustrated, the distal end 126 comprises various internal components 112 enclosed within the body 113. An optical window lens (OWL) 129 is located atop an optical box (OB) 135 at the ultimate extent of the distal end 126, which serves as the interface from which therapeutic light energy is emitted from the probe device 80 during the application of PBMT. In some instances, as shown in FIG. 14, the compliant gasket 134 is included between the OWL 129 and the optical box 135. In some instances, the compliant gasket 134 may comprise a silicone washer. In other instances, the compliant gasket 134 may comprise various other types of gaskets, as desired for a given application. Further, as shown in FIG. 14, the retaining ring 68 is similarly included and is configured to compress the OWL 129 against the compliant gasket 134, which is then compressed against the lens seat 51 to effectively seal the optical box cavity formed within the optical box 135, thereby preventing coolant flowing within the optical box 135 from leaking out from around the OWL 129 during operation.

As illustrated, the OWL 129 has a different shape as compared to the OWL 21. Specifically, the OWL 129 has a beveled outer perimeter that decreases in diameter from the internal surface facing the optical box cavity toward the external surface configured to contact the patient during treatment. The retaining ring 68 is configured to contact this beveled outer perimeter when the retaining ring 68 is threaded onto the optical box 135. However, it should be appreciated that the retaining ring 68 may be shaped, as appropriate, to compress any of a variety of optical window lenses against the compliant gasket 134 and lens seat 51, as desired for a given application. For example, in some instances, the optical window lens utilized could be a single lens, a square convex lens, an oval lens, a double convex oval lens, etc., and the retaining ring 68 may be shaped, as needed, to fit any of these lens types.

One or more external temperature sensors 130 are located adjacent to the OWL 129 within the structure of the body 113 to provide temperature feedback to the CCU 84. The temperature feedback may be communicated via wires 131 connected between the temperature sensor(s) 130 and the CCU 84. In some instances, one or more locating features, such as a pin/hole feature 132 and a rib/groove feature 133 may be configured to aid in aligning the various components which comprise the body 113. As shown, in some instances, the external temperature sensors 130 are arranged on the axial end surface of the distal end 20 of the probe device 1, and may thus be in direct contact with the patient tissue during treatment.

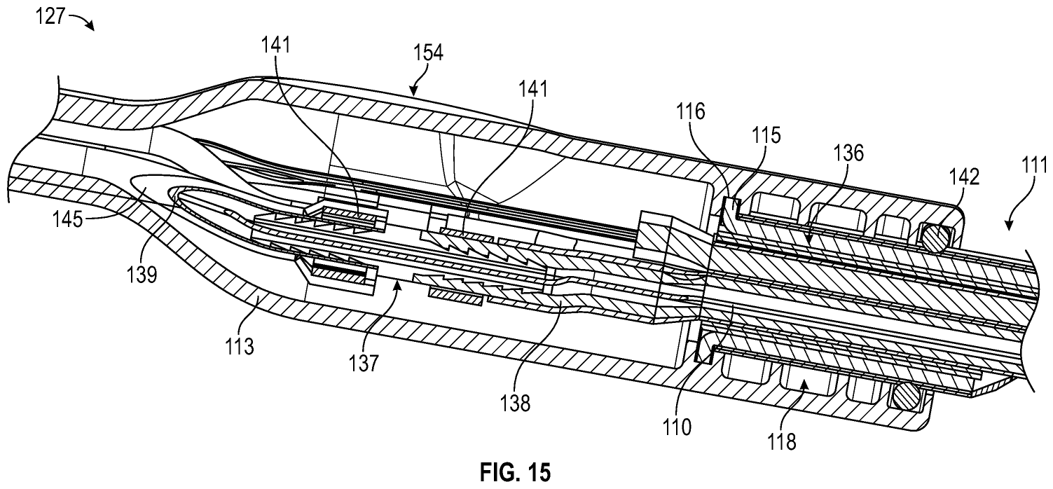

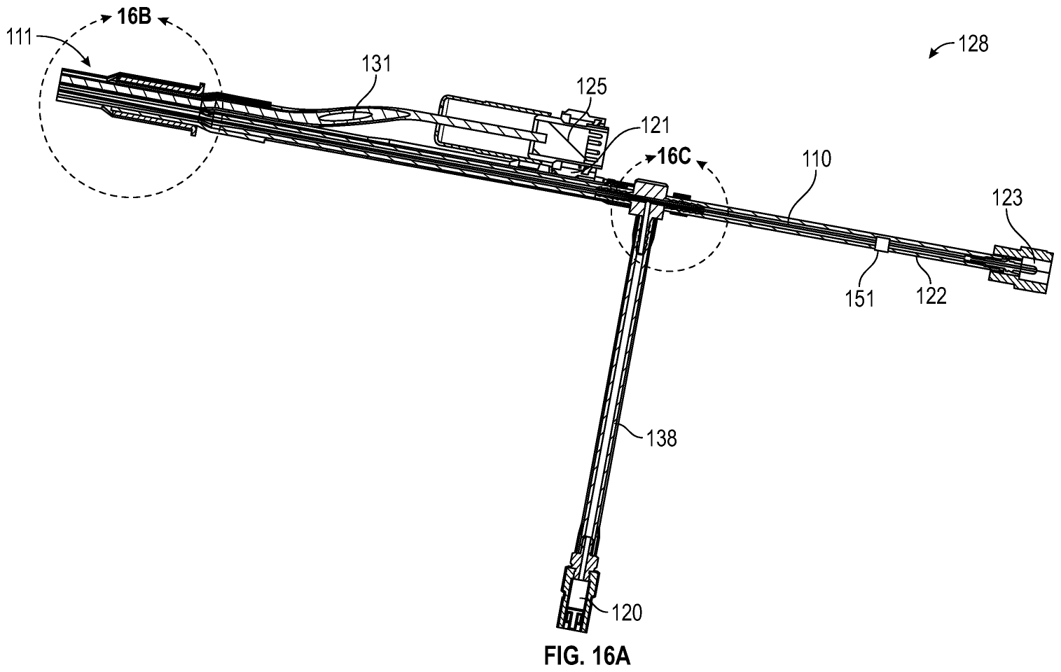

FIG. 15 is a detailed section view of the handle section 127 of the probe device 80. As illustrated, the handle section 127 comprises a probe handle 154 and an umbilical interface 118. The probe handle 154 is a portion of the body 113 that is designed for ergonomic holding and manipulation of the probe device 80 by the end user with one hand. The umbilical interface 118 is a junction apparatus between the assembled probe body 113 and the umbilical cable 111. In some instances, the umbilical interface 118 may comprise or otherwise be fabricated from metal or plastic. In some instances, the umbilical interface 118 may be a single component. In some other instances, the umbilical interface 118 may alternatively comprise an assembly of components.

Various connections 137 are housed within the probe handle 154 portion of the body 113 providing connections between tubing 138 of the umbilical cable 111 and internal tubing 139 of the probe device 80 going to and from the optical box 135. For example, one or more tubing unions 140 are configured to join the tubing 138 of the umbilical cable 111 to the appropriate internal tubing 139. In some instances, the tubing unions 140 may comprise barbed connections. Additionally, the tubing unions 140 may be configured to provide an unobstructed, leak-less connection between the two sections of tubing. Further, in some instances, a crimp ring 141 or other supporting mechanism may be used to reinforce the connection between the tubing union 140 and the tubing 138, 139. In any case, the FOC 110 passes through the incoming tubing 138 of the umbilical cable 111, into the probe handle 154, through a tubing union 140, and into the internal tubing 139, where it continues to the optical box 135 as previously disclosed.

Additionally, in some instances, a sleeve 145 is installed over the tubing 138, 139, the FOC 110, and/or connections (e.g., tubing union 140 or other junction between tubing 139 or other components). The sleeve 145 may be any solid or mesh type encapsulating tubing or wrap configured to insulate, reinforce, or further protect a given connection or tube. For example, in some instances, the sleeve 145 may comprise heat shrink tubing, flexible mesh tubing, expandable rubber tubing, heat wrap insulation, thick foam insulation tubing or wrapping, or any other suitable type of sleeve, as desired for a given application.

As illustrated, a reinforcing support collar 136 within the umbilical interface 118 may be included to provide physical structure/support, locating features, and strain relief for the umbilical cable 111 and body 113 during normal use of the probe device 80. For example, the tubing 138 of the umbilical cable 111 entering the handle section 127 of the body 113 passes through the collar 136. The collar 136 has a rib feature 115, which is received within a mating recess feature or groove 116 within the body 113. The mating features locate the umbilical interface 118 within and with respect to the probe body 113 and serve as a physical strain relief.

A seal 142 may additionally be located within a groove in the probe handle 154 near the end of the physical structure of the body 113. In some instances, the seal 142 may be an o-ring. The seal 142 is configured to serve as a protection mechanism configured to keep foreign contaminants out of the system and to keep the umbilical interface 118 centered within the probe handle 154. In some embodiments, additional sealants (e.g., epoxy) may be used to fill any gaps between the body 113 (e.g., the probe handle 154) and the umbilical interface 118 on either or both sides of the seal 142 to further prevent foreign contaminant intrusion.

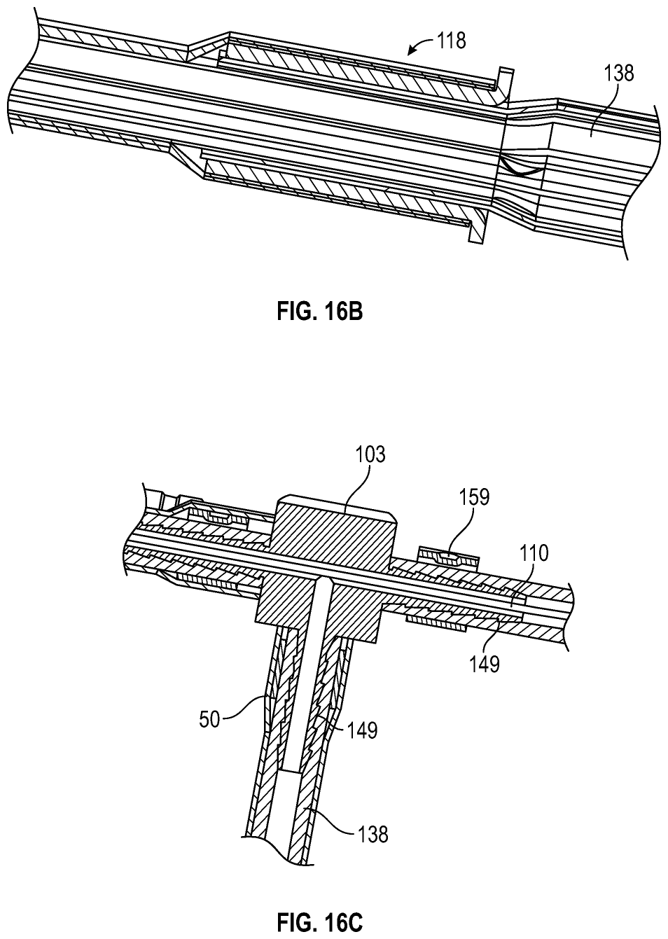

FIG. 16A-16C are various detail views of the proximal end 128 of the umbilical cable 111. The proximal end 128 includes an umbilical interface 143 (shown in FIG. 16B) where the umbilical cable 111 ends, from which the tubing 138 is exposed. The umbilical interface 143 serves to support the end of the umbilical cable 111 and provide a strain relief to the cold-side cooling system connection 121, the hot-side cooling system connection 120, and the CLG connector 123 to the CLG 83 within the enclosure 81 (shown in FIG. 11).

Additionally, wires 131 (e.g., temperature sensor wires) from the probe device 80 are configured to terminated within the multi-pin connector 125 configured for connection directly to the CCU 84 or other data acquisition device (not shown). In some instances, the multi-pin connector 125 could be multiple single pin connecting devices.

As best shown in FIG. 16C, the “T” fitting 103 is configured to facilitate the joining of the cooling system return line (e.g., the hot side 99) and the fiber optic cable 110. In some instances, tubing connections 149 providing connections to and from the “T” fitting 103 are reinforced using crimp-on holders 159 (similar to the crimp rings 141) and covered with a sleeve 150.

The coherent light generator connector 123 is on the end of the fiber optic cable tube 122 with the fiber optic cable 110 extending to and terminating at the CLG connector 123. The fiber optic cable tube 122 may further include a plug 151 within the fiber optic cable tube 122 configured to block the flow of any circulating coolant flowing toward the CLG connector 123, instead forcing the flow of coolant toward the hot side 99 of the cooling system 82.

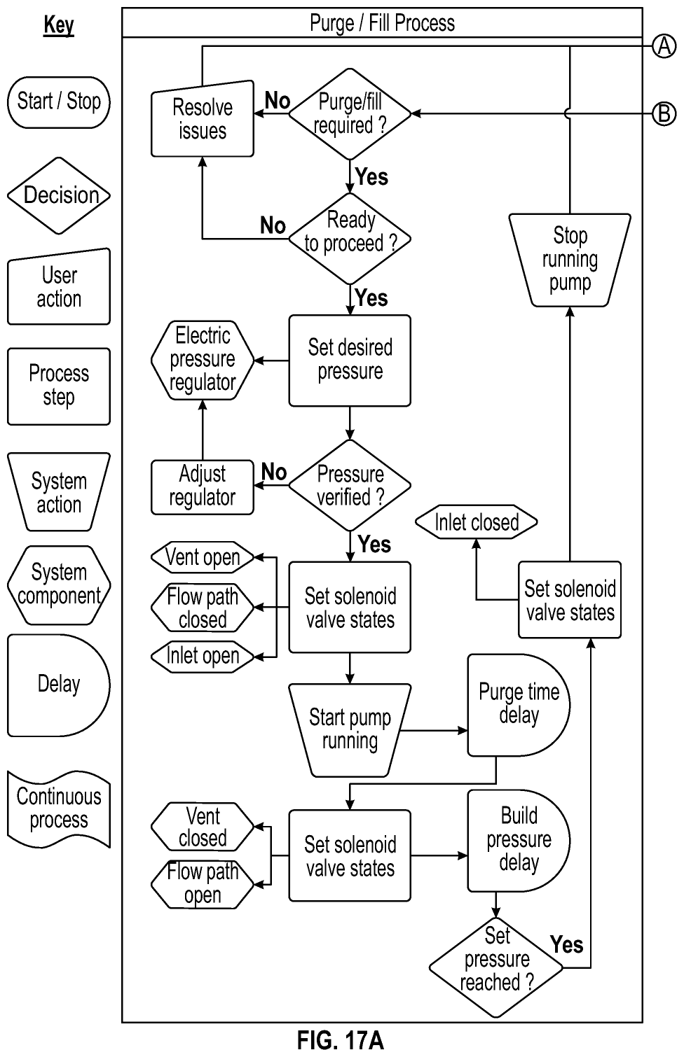

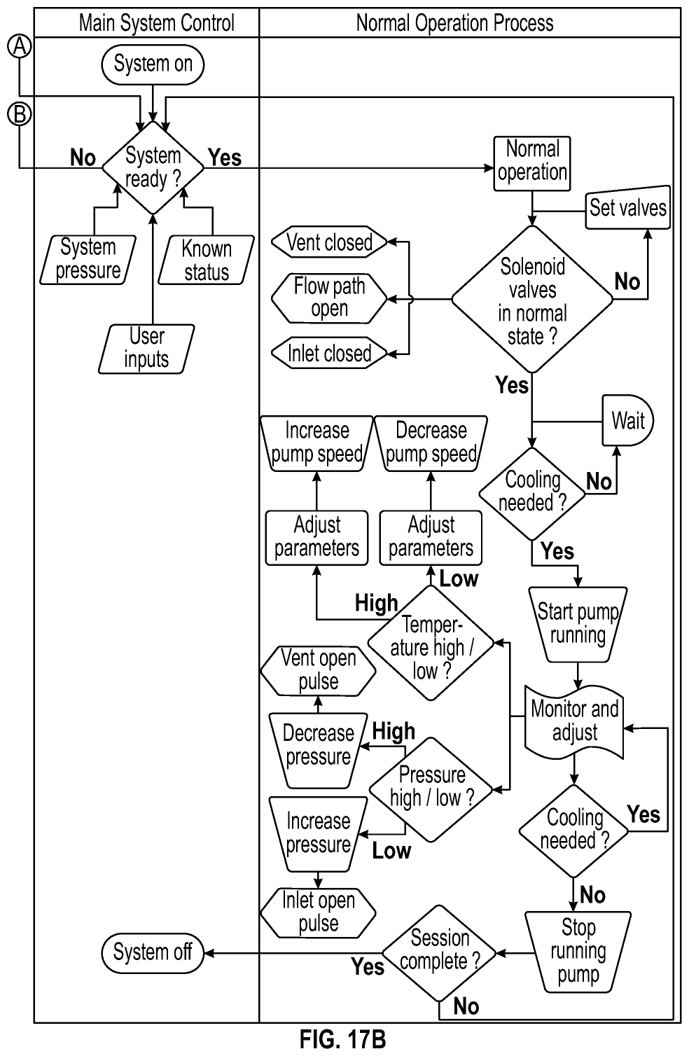

FIGS. 17A and 17B depict a flowchart of a closed-loop cooling system operating scheme for use with the phototherapy system 200, according to an exemplary embodiment. The operating scheme includes differing operating modes and process flows for purging, filling, and normal operation of the closed-loop cooling system. FIG. 17A includes a key on the left-hand side of the diagram that outlines the different flow chart symbols and their representative meanings.

At a high level, three swim lanes sub-divide the various processes into three main control sub-processes. Those sub-processes include a purge/fill process, a main system control process, and a normal operation process. It should be appreciated that various additional sub-processes or process steps not shown may be incorporated into some embodiments and other embodiments may have fewer processes and control elements. Further, although the following description will be in relation to the phototherapy system 200, it should be appreciated that a similar process or scheme may be similarly applied to the phototherapy system 100. Within the following description, text corresponding to the various process flow steps is capitalized to emphasize the process step referred to in the text.

When power is applied and fully initialized (SYSTEM ON), the CCU 84 determines if the cooling system 82 is ready (SYSTEM READY) for cooling of the probe device 80 for the application of PBMT. This determination is based on factors including, but not limited to, the system pressure (SYSTEM PRESSURE—whether the system is pressurized and, if so, what the system pressure is), user inputs (USER INPUTS—is the user doing things that prevent or impact the need for cooling), and various statuses (KNOWN STATUSES—are there errors or other system status messages which need resolved?). If it is determined that the system is not ready (SYSTEM READY=“NO”), the system enters the purge/fill process.

The purge/fill process begins with determining if a purge or fill is required (PURGE/FILL REQUIRED). If a purge or fill is not required (PURGE/FILL=“NO”), then the user needs to complete necessary actions for setup or resolve any errors/messages (RESOLVE ISSUES) to ready the system 82 for operation. Alternatively, if a purge or fill is required (PURGE/FILL REQUIRED=“YES”), the CCU 84 controls the system 82 to progress through the purge/fill process flow with user inputs as needed/directed.

Specifically, if a purge or fill is required, the CCU 84 determines whether the system 82 is ready to proceed with the purge/fill process (READY TO PROCEED). If the system 82 is not ready to proceed (READY TO PROCEED=“NO”) the user needs to resolve any errors/messages to ready the system 82 to proceed (RESOLVE ISSUES). If the system 82 is ready to proceed, (READY TO PROCEED=“YES”), the process continues with the user setting the desired pressure (SET DESIRED PRESSURE). The desired pressure is the target pressure of the closed-loop cooling system 82 to be achieved by the purge/fill process. In some instances, the desired pressure may be controlled by the CCU 84 based on factors including, but not limited to, intended treatment parameters, system settings, or other information inputs. In other instances, the desired pressure may be a user-controlled setting within a pre-specified range. In some instances, the desired pressure setting is sent to the electronic pressure regulator 91 by the CCU 84 (ELECTRONIC PRESSURE REGULATOR). In some other instances, a manually-controlled regulator may be used.

The desired pressure set is then verified (PRESSURE VERIFIED) by an auxiliary pressure sensor (e.g., pressure sensor 92) to ensure the electronic pressure regulator setting matches the set desired pressure. If the electronic pressure regulator setting does not match the set desired pressure, the electronic pressure regulator 91 is then adjusted (ADJUST REGULATOR). In some instances, adjusting the electronic pressure regulator 91 is an automatic action by the CCU 84 to compensate for the difference between the set desired pressure and the electronic pressure regulator setting.

If the electronic pressure regulator setting matches the set desired pressure, the CCU 84 then sets various solenoid valve states (SET SOLENOID VALVE STATES) within the cooling system 82, which may be the initialization action for beginning to execute the purge/fill process of the cooling system 82. For example, the CCU 84 may open the vent path 101 (VENT OPEN) by opening the solenoid valve 95, close the normal coolant flow path (FLOW PATH CLOSED) by closing the solenoid valve 94 to force flow out of the vent 109, and open the coolant inlet path (INLET OPEN) by opening the solenoid valve 93 to allow coolant to enter the cooling system 82.

The CCU 84 may then start running the pump 108 (START RUNNING PUMP) to begin the pumped flow of coolant into and through the system 82 and out of the vent 109. The CCU 84 may continue to run the pump 108 with the system 82 purging for a purge time delay (PURGE TIME DELAY), which is a pre-determined (or adjustable) time delay that the system 82 holds the pump on and vent open to allow any ambient air in the system 82 to be fully purged.

After the purge time delay, the CCU 84 may reset the solenoid valve states (SET SOLENOID VALVE STATES) such that solenoid valve 95 leading to the vent 109 is closed (VENT CLOSED, the solenoid valve 94 in the normal flow path is opened (FLOW PATH OPEN), and the system 82 starts to build pressure. The CCU 84 may allow the system 82 to build pressure for a build pressure delay (BUILD PRESSURE DELAY), which may be a variable delay that continues until the system pressure (as read by the mass flow meter and/or other sensing devices) reaches the set desired pressure.

System pressure is continually checked until the set desired pressure is reached (SET PRESSURE REACHED). Once the set desired pressure is reached, the solenoid valve states are again reset (SET SOLENOID VALVE STATES) such that the solenoid valve 93 is closed (INLET CLOSED), the pump 108 is stopped (STOP RUNNING PUMP), and the system 82 returns to the system ready determination (SYSTEM READY).

Alternatively, if it is determined that the system is ready (SYSTEM READY=“YES”), the normal operation process is entered (NORMAL OPERATION). Once the normal operation process is entered, the CCU 84 may perform a verification that the solenoid valves 93, 94, 95 are in their normal states (SOLENOID VALVES IN NORMAL STATE). That is, the CCU 84 checks whether the solenoid valve 95 is closed (VENT CLOSED), whether the solenoid valve 94 is open (FLOW PATH OPEN) and whether the solenoid valve 93 is closed (INLET CLOSED). If any of the solenoids are not in their normal states (SOLENOID VALVES IN NORMAL STATE=“NO”), the CCU 84 then sets the solenoid valves to the correct states (SET VALVES). Alternatively, if all of the solenoid valves are in their normal states (SOLENOID VALVES IN NORMAL STATE=“YES”), the CCU 84 continues with the normal operation process.

Specifically, the CCU 84 then determines if cooling is currently need by the phototherapy system 200 (COOLING NEEDED). If cooling is not needed (COOLING NEEDED=“NO”), the CCU 84 then holds or waits for cooling to be needed before proceeding (WAIT). If cooling is needed, (COOLING NEEDED—“YES”), the CCU 84 starts to run the pump 108 (START PUMP RUNNING) to begin the circulating flow of coolant within the closed loop cooling system 82 between the heat exchanger 97 and probe device 80.

While running the pump 108, the CCU 84 may be configured to automatically perform a continuous maintenance process of monitoring and adjusting the various components of the cooling system 82 during operation to maintain a set desired pressure and temperature of the probe device 80 (MONITOR AND ADJUST) based on various feedback data (e.g., obtained by various integrated temperatures sensors and/or pressure sensors).

For example, the CCU 84 may determine whether various temperatures within the phototherapy system 200 are too high or too low (TEMPERATURE HIGH/LOW) and to adjust various system parameters if the temperature has gone out of range with respect to set system operation parameters. For example, upon determining that the temperature is too high or low, the CCU 84 may then be triggered to adjust the system parameters (ADJUST PARAMETERS) for the pump 108 by increasing the pump speed (INCREASE PUMP SPEED) to help decrease the temperature when the temperature is high (HIGH) or decreasing the pump speed (DECREASE PUMP SPEED) to help increase the temperature when the temperature is low (LOW).

The CCU 84 may further determine whether various pressures within the phototherapy system 200 are too high or low (PRESSURE HIGH/LOW) and to adjust various system parameters if the pressure deviates from a set desired pressure to adjust the closed-loop cooling system pressure to compensate for the deviation. For example, the CCU 84 may decrease the system pressure (DECREASE PRESSURE) if the system pressure increases beyond the allowable range from the set desired pressure (HIGH), which may occur due to flow restrictions or changes in the environment or coolant. To decrease the pressure, the CCU 84 may perform a vent open pulse (VENT OPEN PULSE) by momentarily opening the solenoid valve 95 to let a small amount of coolant vent from the system. The vent open pulse may be performed once or repeated several times until the set desired pressure is reached.

Alternatively, the CCU 84 may increase the system pressure (INCREASE PRESSURE) if the system pressure decreases beyond the allowable range from the set desired pressure (LOW), which may occur due to small leaks or other environmental or system changes. To increase the pressure, the CCU 84 may perform an inlet open pulse (INLET OPEN PULSE) by momentarily opening the solenoid valve 93 to let a small amount of coolant into the cooling system 82 from the supply tank 88. The inlet open pulse may similarly be performed once or repeated several times until the set desired pressure is reached.

The CCU 84 continues to monitor and adjust the system parameters as long as cooling is needed (COOLING NEEDED=“YES”). However, once the cooling is no longer needed (COOLING NEEDED=“NO”), the CCU 84 is configured to stop running the pump 108 (STOP RUNNING PUMP). The CCU 84 then determines whether the session is complete (SESSION COMPLETE). If the session is complete (SESSION COMPLETE=“YES”), the CCU 84 may then turn the system off (SYSTEM OFF), thereby ending the control scheme. Alternatively, if the session is not complete (SESSION COMPLETE=“NO”), the CCU 84 may then return to the ready state determination step (SYSTEM READY). The control scheme may thus repeat for a subsequent session.

Accordingly, it should be appreciated that the control scheme discussed above allows for the phototherapy system 200 (or the phototherapy system 100) to monitor various real-time temperatures and pressures (both internal and external with respect to the probe device 80 (or the probe device 1)), and to automatically adjust the system pressure and/or the coolant flow rate to effectively maintain a desired pressure and temperature at a variety of locations within the system. Further, although not specifically referenced in FIGS. 17A and 17B, it should be appreciated that, in some instances, the CCU 84 may further be configured to adjust a temperature of the chiller 106 based on various temperature information obtained throughout the system, as desired for a given application.

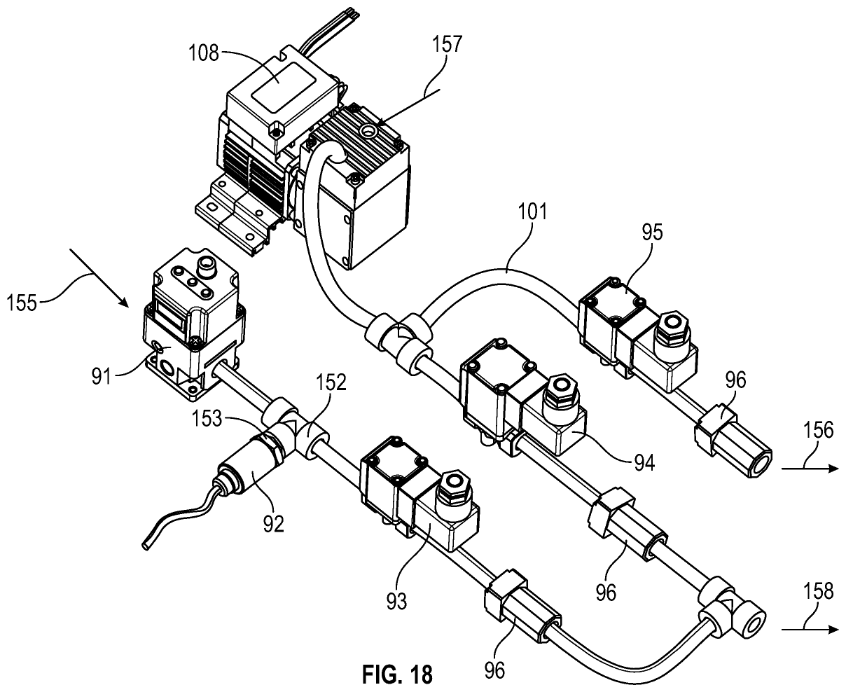

FIG. 18 is a perspective view of an exemplary embodiment of the coolant flow control arrangement described above, with reference to FIG. 11, according to an exemplary embodiment. As depicted, the inlet flow path comprises the electronic pressure regulator 91, the pressure sensor 92, the normally closed solenoid valve 93, and the check valve 96. As illustrated, the pressure sensor 92 may be installed onto a T-fitting 152 in the inlet flow path via an adapter 153. Accordingly, coolant from the supply tank 88 may enter the electronic pressure regulator 91 (as indicated by arrow 155) and flow through the t-fitting 152, the normally closed solenoid valve 93 (when it is selectively opened), and the check valve 96 to charge the system, as discussed above.

The closed-loop flow path includes the coolant pump 108, the normally open solenoid valve 94, and the check valve 96. Accordingly, during normal operation, coolant returning from the probe device 80 enters the pump 108 (as indicated by arrow 157), flows through the normally open solenoid valve 94 and the check valve 96, and flows from the control section depicted in FIG. 18 to the heat exchanger 97 (as indicated by arrow 158). As discussed above, the coolant flowing through the closed-loop flow path may additionally pass through the filter and/or desiccation device 104, the mass flow meter and/or other sensing devices 105 for measuring or conditioning the coolant (e.g., CO₂ gas).

The vent path 101 includes the normally-closed solenoid valve 95 and another check valve 96. Accordingly, when the normally-closed solenoid valve 95 is opened, the coolant can flow freely through solenoid valve 95 and the check valve and out through the vent 109 (shown in FIG. 11) (as indicated by arrow 156).

Accordingly, the various phototherapy systems described herein allow for effective cooling of various optical components of corresponding probe devices. In some instances, this effective cooling may be provided by the inclusion of a coolant flow path that allows for coolant (e.g., CO₂ gas) to be provided to an optical box of the handheld probe to cool the components of the optical box. In these instances, the coolant flow path may allow for coolant exhausted through various channels formed in the optical box and corresponding fiber connection components to effectively flow along and cool the optical box outlets, the diffusing lens (e.g., the ball lens), the corresponding fiber connection components, and the fiber optic cable as the coolant is exhausted into a collection sleeve and ultimately out of the probe device. In some other instances, the various components of the probe devices may be achieved without a coolant flow path that flows directly through the optical box.

For example, it should be appreciated that, although particular configurations have been described herein, various changes and/or alterations to the systems discussed above may be implanted without departing from the scope of the present disclosure. For instance, although the phototherapy systems described herein described the coolant flowing in a particular direction (e.g., in through the inlet of the optical box and exiting through the vent holes and collection sleeve), in some instances, the coolant flow direction could be reversed, as desired for a given application.

Additionally, in some instances, the coolant flow path within the various probes discussed herein may be modified and/or various additional cooling features may be utilized, as desired for a given application. For example, as shown in FIG. 3C, in some instances, the probe device 1 may additionally or alternatively include one or more lens cooling features 74, one or more optical box cooling features 75, one or more embedded distal end cooling features 76, and/or one or more external cooling features 77.

In some instances, the various cooling features 74, 75, 76, 77 may comprise additional coolant paths that are in fluid communication with the coolant flow path of the probe device 1 to allow for coolant to flow through and provide cooling directly to the optical window lens 21, the optical box 30, the distal end 20 of the probe device 1, and/or to an external area adjacent the probe device 1 during treatment. For example, in some instances, one or more inlet fluid paths may extend from the internal tubing 43 to any or all of the various cooling features 74, 75, 76, 77, and one or more outlet fluid paths may extend from any or all of the various cooling features 74, 75, 76, 77 to the coolant collection space 67. As such, during operation, in some instances, coolant may flow from the internal tubing 43, through the one or more inlet fluid paths to any or all of the various cooling features 74, 75, 76, 77. The coolant may then flow from any or all of the various cooling features 74, 75, 76, 77, through the one or more outlet fluid paths to the coolant collection space 67. In some instances, the fluid paths connecting the various cooling features to the internal tubing 43 and the coolant collection space 67 may allow for the coolant to completely bypass the optical box cavity 31. As such, in some instances, the coolant inlet port 38 may be omitted and coolant may thus be prevented from entering the optical box cavity 31 during operation. The prevention of coolant from entering the optical box cavity 31 may be beneficial for optical transmission through the optical box cavity 31 when a liquid coolant is utilized instead of a gaseous coolant.

In some instances, the one or more lens cooling features 74 may comprise one or more fluid paths embedded within the optical window lens 21. For example, in some instances, the lens cooling features 74 may comprise a fluid path embedded within and extending around a perimeter of the optical window lens 21. Although depicted as embedded features, in some instances, the one or more lens cooling features 74 may further comprise one or more external lens cooling features (e.g., similar to the external cooling features 77) that are affixed to an external surface of the optical window lens 21 (e.g., around a perimeter of the optical window lens 21). In either case, the lens cooling features 74 may directly cool the optical window lens 21 and, in some instances, directly cool patient tissue during treatment (e.g., in the case of the external lens cooling features).

In some instances, the one or more optical box cooling features 75 may comprise one or more fluid paths embedded within the optical box 30. For example, in some instances, the optical box cooling features 75 may extend within the sidewalls of the optical box 30 around a circumference of the optical box 30 to allow for cooling of the sidewalls of the optical box 30. In some instances, the optical box cooling features 75 may further extend into the lower portion of the optical box 30 (e.g., the portion of the optical box 30 including the fiber ferrule bore 70) to provide cooling to the fiber ferrule 33, the FOC 14, and/or the diffusing lens 32 during operation.

In some instances, the one or more embedded distal end cooling features 76 may comprise one or more fluid paths embedded within the distal end 20 of the probe device 1. For example, in some instances, the embedded distal end cooling features 76 may extend within the sidewalls of the distal end 20 around a circumference of the distal end 20 to allow for cooling of the sidewalls of the distal end 20. It should be appreciated that, although the embedded distal end cooling features 76 are shown included within only the distal end 20 of the probe device 1, in some instances, the embedded distal end cooling features 76 may extend throughout the shaft 162 to additionally provide cooling along an axial length of the shaft 162, as desired for a given application.

In some instances, the one or more external cooling features 77 may comprise one or more fluid paths affixed to an external surface of the distal end 20 of the probe device 1. For example, in some instances, the external cooling features 77 may extend around an external circumference of the distal end 20. Although depicted on a radially outward facing surface of the distal end 20, in some instances, the external cooling features 77 may further extend onto the axial end surface (i.e., the surface including the external surface of the optical window lens 21 and the external temperature sensors 28) of the distal end 20, around at least a portion of a perimeter of the optical window lens 21, to provide direct cooling to the patient tissue during treatment.

Furthermore, in the case that the probe device 1 is used to deliver transcutaneous light therapy to the skin of the patient, in some instances, one or more of the external cooling features 77 may be configured to discharge coolant (e.g., CO₂ gas) onto the surface of the patient's skin to directly cool the tissue during treatment. In some of these instances, the one or more external cooling features 77 may receive coolant via the same cooling system (e.g., the cooling system 9) or a separate cooling system. In some instances, directly cooling the patient's skin may allow for a higher intensity light therapy treatment as compared to solely internal cooling within the probe device 1. Furthermore, in some instances, one or more of the external cooling features 77 configured to discharge coolant onto the surface of the patient's skin may have a controllable external opening (e.g., controllable via the CCU 10) that allows for the selective discharge of coolant (e.g., to prevent discharge of coolant when the probe device 1 is utilized within a patient cavity).

Additionally or alternatively, in some instances, any or all of various cooling features 74, 75, 76, 77 may instead comprise one or more thermoelectric cooling elements configured to thermoelectrically cool the optical window lens 21, the optical box 30, the distal end 20 of the probe device 1, and/or to an external area adjacent the probe device 1 during treatment. For example, in some instances, any of the various thermoelectric cooling elements may be in communication with the integrated electronics 16, and may thus be wirelessly controlled by the CCU 10 (or the CCU 84) to actively cool the various components of the probe device 1 during operation.

In some instances, as shown in FIG. 3A, an external chilling blanket 78 may be utilized with the probe device 1 to provide external cooling to the distal end 20 and the shaft 162 of the probe device 1, as well as direct cooling to the patient tissue during treatment. As illustrated, in some instances, the external chilling blanket 78 may be configured to be slid over the distal end 20 and the shaft 162 of the probe device 1. In some instances, the external chilling blanket 78 may similarly be cooled using a flowing coolant passing through various fluid channels within the external chilling blanket 78. In some other instances, the external chilling blanket 78 may be chilled using one or more thermoelectric cooling elements. Further, in some instances, the external chilling blanket 78 may include an aperture configured to align with the optical window lens 21 to allow for the light therapy to be unobstructed during treatment.

In some instances, the coolant itself may be utilized to direct the laser energy in addition to providing cooling during treatment. For example, in some instances, the light therapy (e.g., the coherent light) may be guided through an internally reflective coolant path (e.g., an internally reflective coolant tube) that is used in place of the FOC 14 (or a distal portion thereof). In some of these instances, a coolant bypass channel 79 may be included between the internal tubing 43 and the distal end of the internally reflective coolant tube (e.g., as shown in FIG. 3C) to allow the coolant to flow from the internal tubing 43, through the coolant bypass channel 79, and into the distal end of the internally reflective coolant tube without entering the optical box cavity 31. As such, in some instances, the probe device 1 may not include the FOC 14.

Additionally, although the various cooling systems described herein have been discussed and shown in reference to phototherapy systems utilizing handheld phototherapy devices, it should be appreciated that, in some embodiments, the cooling systems described herein may be implemented within various other light treatment, phototherapy, PBMT, low level light therapy, photodynamic therapy, and/or laser physiotherapy systems utilizing various other handheld devices, probes, hand pieces, and/or other therapy devices (e.g., non-handheld devices), as desired for a given application. For example, in some instances, the various cooling systems described herein may be implemented within PBMT systems to cool endoscopic and/or endoluminal PBMT devices or apparatuses.

As described herein, one embodiment of the present disclosure relates to a system for administering phototherapy comprising a cooling system, a coherent light generator, and a therapy probe device. The cooling system is configured to selectively circulate a coolant. The coherent light generator is configured to produce a beam of coherent light. The therapy probe device includes an optical box at a distal end of a shaft. The therapy probe device further includes a fiber optic cable extending through the shaft to the optical box and configured to transmit the beam of coherent light from the coherent light generator through the fiber optic cable, into the optical box which diffuses the beam, and out through an optical window lens at a tip of the therapy probe device. The therapy probe device further includes a coolant flow path. The coolant flow path originates within flow rate generation mechanisms or components of a cooling system and continues and extends through an incoming coolant exhausting tube, through a shaft of the therapy probe device, and into and through a chamber of the optical box. The coolant flow path at least partially envelops the fiber optic cable.

The present disclosure contemplates methods, systems, and program products on any machine-readable media for accomplishing various operations. The embodiments of the present disclosure may be implemented using existing computer processors, or by a special purpose computer processor for an appropriate system, incorporated for this or another purpose, or by a hardwired system. Embodiments within the scope of the present disclosure include program products comprising machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, CD-ROM or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. When information is transferred or provided over a network or another communications connection (either hardwired, wireless, or a combination of hardwired or wireless) to a machine, the machine properly views the connection as a machine-readable medium. Thus, any such connection is properly termed a machine-readable medium. Combinations of the above are also included within the scope of machine-readable media. Machine-executable instructions include, for example, instructions and data which cause a general purpose computer, special purpose computer, or special purpose processing machines to perform a certain function or group of functions.

Although the figures may show a specific order of method steps, the order of the steps may differ from what is depicted. Also two or more steps may be performed concurrently or with partial concurrence. Such variation will depend on the software and hardware systems chosen and on designer choice. All such variations are within the scope of the disclosure. Likewise, software implementations could be accomplished with standard programming techniques with rule based logic and other logic to accomplish the various connection steps, processing steps, comparison steps and decision steps.

Also, the term “or” is used in its inclusive sense (and not in its exclusive sense) so that when used, for example, to connect a list of elements, the term “or” means one, some, or all of the elements in the list. Conjunctive language such as the phrase “at least one of X, Y, and Z,” unless specifically stated otherwise, is otherwise understood with the context as used in general to convey that an item, term, etc. may be either X, Y, Z, X and Y, X and Z, Y and Z, or X, Y, and Z. Thus, such conjunctive language is not generally intended to imply that certain embodiments require at least one of X, at least one of Y, and at least one of Z to each be present.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed:

1. A system for administering phototherapy comprising:
a cooling system configured to selectively circulate a coolant;
a coherent light generator configured to produce a beam of coherent light; and
a probe device including an optical box at a distal end of a shaft, a fiber optic cable extending through the shaft to the optical box and configured to transmit the beam of coherent light from the coherent light generator to the optical box, and a coolant flow path extending through the shaft to the optical box and at least partially enveloping the fiber optic cable.

2. The system of claim 1, wherein the coolant flow path includes a coolant inlet flow path configured to deposit the coolant into the optical box and a coolant outlet flow path extending through a proximal end of the optical box and at least partially enveloping the fiber optic cable.

3. The system of claim 2, wherein the coolant outlet flow path comprises at least one vent port disposed adjacent to a diffusing lens arranged at or near the proximal end of the optical box, the coolant outlet flow path being configured to allow the coolant to directly cool the diffusing lens.

4. The system of claim 1, wherein the probe device further includes at least one of an internal temperature sensor or an external temperature sensor, and the cooling system is configured to selectively adjust at least one of a flow rate, a pressure, or a temperature of the coolant based on feedback from the at least one of the internal temperature sensor or the external temperature sensor.

5. The system of claim 4, wherein the probe device includes the external temperature sensor and the external temperature sensor is arranged adjacent to an emission lens of the probe device.

6. The system of claim 1, wherein the coolant is a gas comprising one of CO₂, nitrogen, or air.

7. The system of claim 1, wherein the coolant is a liquid and the coolant flow path extends through at least one of a sidewall of the optical box, a sidewall of a distal end of the probe device, or an emission lens of the probe device.

8. The system of claim 1, further comprising an external chilling blanket configured to be slid over a distal end and the shaft of the probe device.

9. The system of claim 8, wherein the external chilling blanket is cooled via one of a flowing coolant or a thermoelectric cooling element.

10. The system of claim 1, wherein the probe device further comprises one or more thermoelectric cooling elements embedded within or affixed to one or more of an emission lens, the optical box, or the distal end of the probe device.

11. A system for administering phototherapy comprising:
a cooling system configured to selectively circulate a coolant;
a coherent light generator configured to produce a beam of coherent light; and
a probe device including an optical box at a distal end of a shaft and a coolant flow path extending through the shaft to the optical box, the optical box being configured to transmit the beam of coherent light from the coherent light generator through an emission lens, and the coolant flow path including a coolant inlet flow path configured to deposit the coolant into the optical box and a coolant outlet flow path configured to allow the coolant to exit the optical box.

12. The system of claim 11, wherein the probe device further includes at least one of an internal temperature sensor or an external temperature sensor, and the cooling system is configured to selectively adjust at least one of a flow rate, a pressure, or a temperature of the coolant based on feedback from the at least one of the internal temperature sensor or the external temperature sensor.

13. The system of claim 12, wherein the probe device includes the external temperature sensor and the external temperature sensor is arranged adjacent to the emission lens of the probe device.

14. The system of claim 11, wherein the coolant flow path is an internal coolant flow path and the cooling system includes an external coolant flow path, and wherein at least a portion of at least one of the internal coolant flow path or the external coolant flow path includes an insulating sleeve.

15. The system of claim 11, wherein the probe device further comprises one or more thermoelectric cooling elements embedded within or affixed to one or more of the emission lens, the optical box, or the distal end of the probe device.

16. The system of claim 11, wherein the coolant outlet flow path comprises at least one vent port disposed adjacent to a diffusing lens arranged at or near a proximal end of the optical box, the coolant outlet flow path being configured to allow the coolant to directly cool the diffusing lens.

17. A probe device for administering phototherapy, the probe device comprising:
an optical box at a distal end of a shaft;
a fiber optic cable extending through the shaft to the optical box and configured to transmit a beam of coherent light from a coherent light generator through the optical box; and
a coolant flow path extending through the shaft to the optical box and at least partially enveloping the fiber optic cable.

18. The probe device of claim 17, wherein the coolant flow path includes a coolant inlet flow path configured to deposit coolant into the optical box and a coolant outlet flow path extending through a proximal end of the optical box and at least partially enveloping the fiber optic cable.

19. The probe device of claim 18, wherein the coolant outlet flow path comprises at least one vent port disposed adjacent to a diffusing lens arranged at or near the proximal end of the optical box, the coolant outlet flow path being configured to allow the coolant to directly cool the diffusing lens.

20. The probe device of claim 17, further including an external temperature sensor arranged adjacent to an emission lens of the probe device.
